(19) United States
(12) Patent Application Publication
(10) Pub. No.: US 2019/0282616 A1
(43) Pub. Date: Sep. 19, 2019

(54) MACROPHAGES REDIRECT PHAGOCYTOSIS BY NON-PROFESSIONAL PHAGOCYTES AND INFLUENCE INFLAMMATION

(71) Applicant: University of Virginia Patent Foundation, Charlottesville, VA (US)

(72) Inventors: Kodimangalam S. Ravichandran, Earlysville, VA (US); Claudia Zhuyun Han, San Diego, CA (US)

(73) Assignee: University of Virginia Patent Foundation, Charlottesville, VA (US)

(21) Appl. No.: 16/347,752

(22) PCT Filed: Nov. 3, 2017
(86) PCT No.: PCT/US2017/059915

§ 371 Date: May 6, 2019

Related U.S. Application Data
(60) Provisional application No. 62/418,469, filed on Nov. 7, 2016.

(51) Int. Cl. A61K 35/12; A61K 38/20; C07K 14/52; C07K 14/65; G01N 33/68; A61K 38/17; A61K 9/127

(52) U.S. Cl. CPC A61K 35/12; A61K 38/2086; A61K 38/2026; A61K 9/127; C07K 14/65; G01N 33/68; A61K 38/1709; C07K 14/52

(57) ABSTRACT

Professional phagocytes (such as macrophages) and non-professional phagocytes (such as epithelial cells) clear billions of apoptotic cells and particles on a daily basis. Since these phagocytes reside in proximity in most tissues, whether cross-communication exists between them during cell clearance, and how this might impact inflammation are not known. Here, we show that macrophages, via the release of a soluble growth factor and microvesicles, redirect the type of particles engulfed by non-professional phagocytes and influence their inflammatory response. During apoptotic cell engulfment or in response to inflammation-associated cytokines, macrophages released insulin-like growth factor 1 (IGF-1). The binding of IGF-1 to its receptor on non-professional phagocytes redirected their phagocytosis, such that uptake of larger apoptotic cells was dampened while engulfment of microvesicles was enhanced. Macrophages were refractory to this IGF-1 mediated engulfment modulation. Macrophages also released microvesicles, whose uptake by epithelial cells, enhanced by IGF-1, led to decreased inflammatory responses by epithelial cells. Consistent with these observations, deletion of IGF-1 receptor in airway epithelial cells led to exacerbated lung inflammation after allergen exposure. These genetic and functional studies reveal a novel IGF-1 and microvesicle-dependent communication between macrophages and epithelial cells that can critically influence the magnitude of tissue inflammation in vivo.

### CROSS REFERENCE TO RELATED APPLICATIONS

This application is entitled to priority pursuant to 35 U.S.C. § 119(e) to U.S. provisional patent application No. 62/418,469 filed Nov. 7, 2016. The entire disclosure of the afore-mentioned patent application is incorporated herein by reference.

### STATEMENT REGARDING FEDERALLY SPONSORED RESEARCH OR DEVELOPMENT

This invention was made with government support under Grant Nos. GM064709, MH096484, HL120840, HL132287, and HL091127, awarded by The National Institutes of Health. The government has certain rights in the invention.

### BACKGROUND

It has long been known that professional and non-professional phagocytes reside in proximity and can engulf dying cells and debris. The mechanisms controlling these processes and how to regulate inflammation are largely unknown. Although professional and non-professional macrophages reside in proximity in most tissues, whether they communicate with each other during cell clearance, and how this might affect inflammation, is not known.

There is a long felt need in the art for compositions and methods for regulating inflammation and inflammation associated responses of non-professional phagocytes and for regulating the interactions between macrophages and non-professional phagocytes. The present invention satisfies these needs.

### SUMMARY OF THE INVENTION

The data disclosed herein provide new insights into phagocytosis and tissue inflammation, including identification of a rapid, transient, and reversible regulation, wherein soluble Insulin-like Growth Factor I (IGF-I) from macrophages influences the type of particle uptake by epithelial cells and the soluble IGF-I stimulates the release of microvesicles from the macrophages. In one aspect, the soluble IGF-I stimulates microvesicle uptake by non-professional phagocytes. The present application further discloses a two-part regulation of epithelial cells by macrophages which impacts airway inflammation, i.e., the secretion of IGF-I that redirects particle uptake, and the release of microvesicles that dampen inflammatory cytokine production by epithelial cells. Also disclosed is the unexpected result that the IGF-IR is required on the target non-professional phagocyte for this to occur.

To further address the problem, in order to investigate insulin-like growth factor I (also referred to as IGF-I and IGF-1)/insulin-like growth factor I receptor (IGF-I/IGF-IR) signaling in inflammation, one model used was the model of airway inflammation induced by the allergen house dust mite (HDM) where apoptotic cell recognition by airway epithelial cells influences inflammation.

In one embodiment, the present invention provides a method for decreasing an inflammatory response in non-professional phagocytes in a subject in need thereof. In one aspect, the method comprises administering to the subject a pharmaceutical composition comprising an effective amount of macrophage-derived microvesicles or it comprises stimulating macrophages in the subject to secrete Insulin-like Growth Factor-I (IGF-I) and to release microvesicles. In one aspect, macrophage-derived microvesicles are administered and compounds are administered as a combination to stimulate macrophage microvesicle release from endogenous macrophages.

In one embodiment, uptake of the microvesicles by the non-professional phagocytes decreases synthesis and release of one or more inflammatory response associated proteins by non-professional phagocytes. In one aspect, and the IGF-I secreted by macrophages enhances uptake of the microvesicles into the non-professional phagocytes. In one aspect, the inflammatory response of non-professional phagocytes is decreased.

In one aspect, the non-professional phagocytes being targeted by the compositions and methods of the invention are epithelial cells. In one aspect, the epithelial cells are airway epithelial cells.

In one aspect, the inflammatory response of the airway epithelial cells being targeted by the compositions and methods of the invention is initiated by an allergen contacting the airway epithelial cells. The allergen can be, for example, house dust mites (HDM).

In one embodiment, the inflammatory response associated proteins are selected from the group consisting of thymic stromal lymphopoietin (TSLP), colony stimulating factor 2/granulocyte-macrophage colony stimulating factor (CSF-2/GM-CSF), interleukin 6 (IL-6), interleukin 8 (IL-8), interleukin 25 (IL-25), interleukin 33 (IL-33), fibroblast growth factor 2 (FGF2), Kruppel-like factor 4 (KLF4), Interferon-induced Protein with Tetratricopeptide Repeats 2 (IFIT2), and Pentraxin 3 (PTX3).

In one embodiment, an effective amount of Interleukin-4 (IL-4) or Interleukin-13 (IL-13), or biologically active fragments or homologs of either protein, is administered to the subject to stimulate the macrophages to release macrophage vesicles and IGF-I. In one aspect, both IL-4 and IL-13, or biologically active fragments or homologs of the proteins, are administered.

In one aspect, the compositions and methods of the invention stimulate an increase in macrophage microvesicle uptake by the non-professional phagocytes.

In one aspect, the compositions and methods of the invention are useful for inhibiting uptake of apoptotic cells by the non-professional phagocytes. In one aspect, macrophage-derived microvesicles administered to the subject are purified macrophage-derived microvesicles.

In one aspect, an effective amount of IGF-1 is administered to a subject to increase macrophage microvesicle uptake by the non-professional phagocytes, whether the macrophages are stimulated in situ or macrophage-derived.

In one aspect, the inflammatory response is inflammatory cytokine production.

The present invention also provides compositions and methods for decreasing an inflammatory response in non-professional phagocytes in a subject in need thereof. In one aspect, the method comprises administering to the subject a pharmaceutical composition comprising an effective amount of Insulin-like Growth Factor I (IGF-I), or biologically active fragments or homologs thereof, macrophage-derived microvesicles, insulin, or biologically active fragments or homologs thereof, or Insulin-like Growth Factor-II, or biologically active fragments or homologs thereof, or a combination thereof. In one aspect, the decreases uptake of apoptotic cells by the non-professional phagocytes. In one aspect, the method increases the uptake of macrophage microvesicles. In one aspect, the decrease in the inflammatory response in the non-professional phagocytes is a decrease in the synthesis, levels, or release of one or more inflammatory response associated proteins in the non-professional phagocytes.

In one aspect, inflammatory response associated proteins that are regulated in target non-professional phagocytes of the invention, include, but are not limited to, thymic stromal lymphopoietin (TSLP), colony stimulating factor 2/granulocyte-macrophage colony stimulating factor (CSF-2/GM-CSF), interleukin 6 (IL-6), interleukin 8 (IL-8), interleukin 25 (IL-25), interleukin 33 (IL-33), fibroblast growth factor 2 (FGF2), Kruppel-like factor 4 (KLF4), Interferon-Induced Protein with Tetratricopeptide Repeats 2 (IFIT2), and Pentraxin 3 (PTX3).

In one aspect, an effective amount of IL-4, or biologically active fragments or homologs thereof, is administered to the subject to stimulate macrophages to release macrophage vesicles and

In one aspect, the compositions and methods of the invention stimulate an increase in macrophage microvesicle uptake by non-professional phagocytes.

In one aspect, when macrophage-derived microvesicles are administered to the subject they are purified before administration.

In one aspect, an effective amount of IGF-I is administered to a subject to increase macrophage microvesicle uptake by non-professional phagocytes.

In one aspect, uptake of the microvesicles by non-professional phagocytes decreases at least one of synthesis, levels, and release of one or more inflammatory response associated proteins by the non-professional phagocytes.

In one aspect, the non-professional phagocytes being targeted are epithelial cells.

In one aspect, the epithelial cells being targeted are airway epithelial cells. In one aspect, the type of inflammatory response being treated is one that is initiated by an allergen contacting the airway epithelial cells.

In one aspect, the method decreases the increase in cytokine synthesis associated with the inflammatory response in the target non-professional phagocytic cells.

The present application further discloses a method to decrease an inflammatory response in an epithelial cell comprising contacting the epithelial cell with an effective amount of Insulin-like Growth Factor I (IGF-I), or biologically active fragments or homologs thereof, macrophage-derived microvesicles, insulin, or biologically active fragments or homologs thereof, or Insulin-like Growth Factor-II, or biologically active fragments or homologs thereof, or a combination thereof, wherein the method decreases uptake of apoptotic cells. In one aspect, uptake of macrophage-derived microvesicles by the epithelial cell is increased by contacting the epithelial cell with IGF-I. In one aspect, the epithelial cell is an airway epithelial cell. In one aspect, the inflammatory response is initiated by exposure of the epithelial cell to an allergen. In one aspect when macrophage-derived microvesicles are used they are purified, and then the epithelial cell is contacted with the purified macrophage-derived microvesicles. In one aspect, macrophage-derived microvesicles have been released by macrophages stimulated to release the microvesicles. In one aspect, the macrophages are stimulated to release the microvesicles by contacting the macrophages with Interleukin-4. In one aspect, the macrophages are in close proximity to the epithelial cell.

In one aspect, IGF-I is used at dosages to achieve a blood level of about 100 to about 600 ng/ml. In one aspect, IGF-I is used at dosages to achieve a blood level of about 500 to about 1,000 ng/ml. In one aspect, IGF-I is used at dosages to achieve a blood level of about 800 to about 2,000 ng/ml.

In one embodiment, macrophages are stimulated in vivo to release IGF-I by exposure to IL-4, IL-13, or biologically active fragments or homologs thereof, and/or the presence of apoptotic cells. In one aspect, an effective of amount of IL-4, IL-13, or biologically active fragments or homologs thereof, or a combination of the two is administered to a subject in need thereof to stimulate release of IGF-I by macrophages.

In one embodiment, the macrophage is an alveolar macrophage.

In one embodiment, the compositions and methods of the invention are useful for preventing or treating inflammatory responses to allergens in the lungs.

In another embodiment, the compositions and methods of the invention are useful for treating inflammation due to smoking-induced lung injury.

In one embodiment, the macrophages and the non-professional phagocytes are in close proximity to one another, such as in the same tissue. In one aspect, the cells are in the lung.

In one embodiment, the present invention is useful for enhancing the uptake of particles smaller than apoptotic cells into non-professional phagocytes. In one aspect, the smaller particles are liposomes.

In one embodiment, the macrophage microvesicles of the invention range in size from about 50 to about 2,000 nm. In one aspect, they range in size from about 100 to about 1,500 nm. In one aspect, they range in size from about 200 to about 1,200 nm. In one aspect, they range in size from about 100 to about 1,000 nm. In one aspect, they range in size from about 200 to about 800 nm. In one aspect, they range in size from about 300 to about 900 nm. In one aspect, they range in size from about 400 to about 600 nm.

In one embodiment, the present invention provides compositions and methods for decreasing an inflammatory response in non-professional phagocytes. In one aspect, the non-professional phagocyte is an epithelial cell. In one aspect, the epithelial cell is an airway epithelial cell. In one aspect, the invention provides compositions and methods for decreasing the production of one or more inflammatory response associated proteins by a non-professional phagocyte. In one aspect, the invention provides compositions and methods for decreasing the synthesis and release of one or more inflammatory response associated proteins by a non-professional phagocyte. In one aspect, the invention provides compositions and methods for decreasing the release of one or more inflammatory response associated proteins by a non-professional phagocyte.

In one aspect, the inflammatory response associated proteins, include, but are not limited to, thymic stromal lymphopoietin (TSLP), colony stimulating factor 2/granulocyte-macrophage colony stimulating factor (CSF-2/GM-CSF), interleukin 6 (IL-6), interleukin 8 (IL-8), interleukin 25 (IL-25), interleukin 33 (IL-33), fibroblast growth factor 2 (FGF2), Kruppel-like factor 4 (KLF4). Interferon-Induced Protein with Tetratricopeptide Repeats 2 (IFIT2), and Pentraxin 3 (PTX3).

In one embodiment, a composition of the invention is administered based on the type of inflammation to be treated, the location of the cells to be treated, etc. In one aspect, a composition of the invention is administered intranasally.

In one embodiment, a pharmaceutical composition of the invention can comprise at least one additional therapeutic agent, such as an antimicrobial agent, an additional anti-inflammatory agent, etc.

The invention further encompasses a kit for use in treating or preventing an inflammatory response in a subject in need thereof. The kit can comprise one of more proteins of the invention, purified macrophage microvesicles, an applicator, and an instructional material for the use thereof.

Various aspects and embodiments of the invention are described in further detail below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1, comprising FIGS. 1a-1g, demonstrates that IGF-I (also referred to as IGF-1) dampens apoptotic cell engulfment and enhances liposome uptake by non-professional phagocytes.

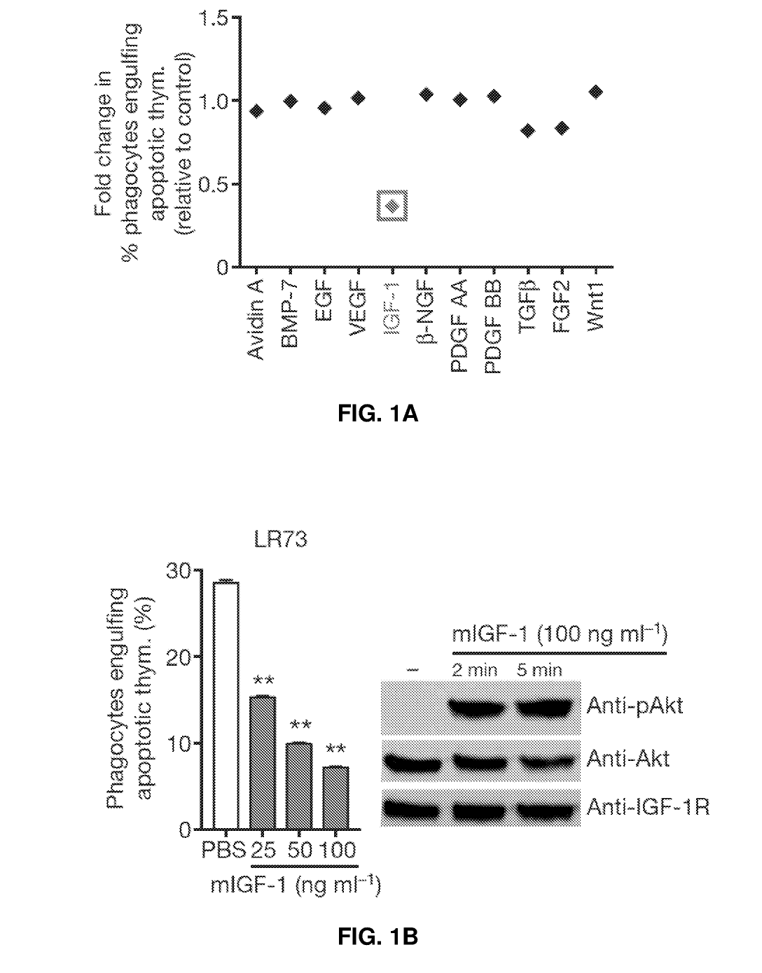

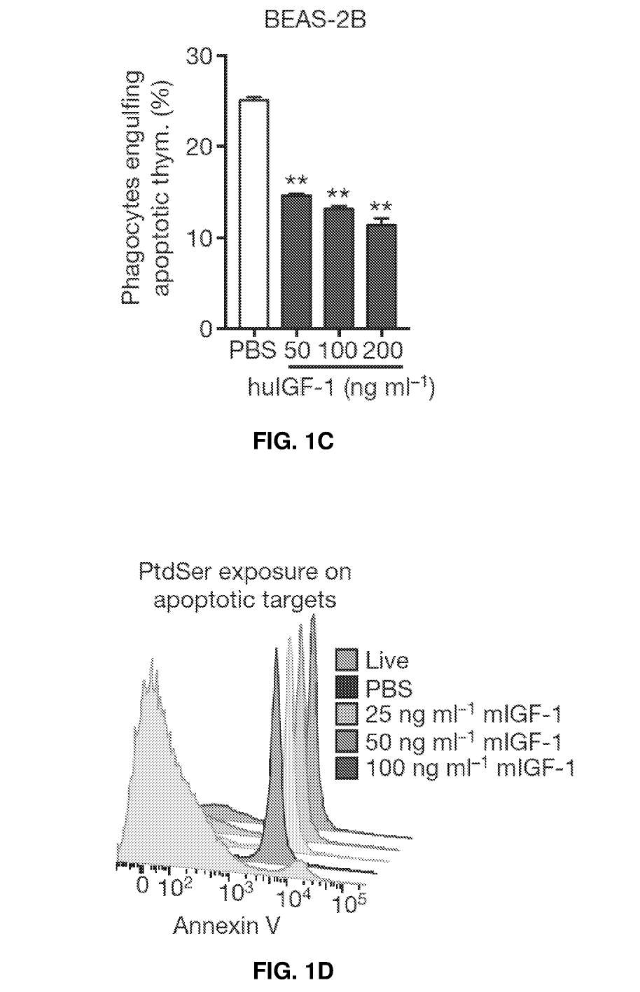

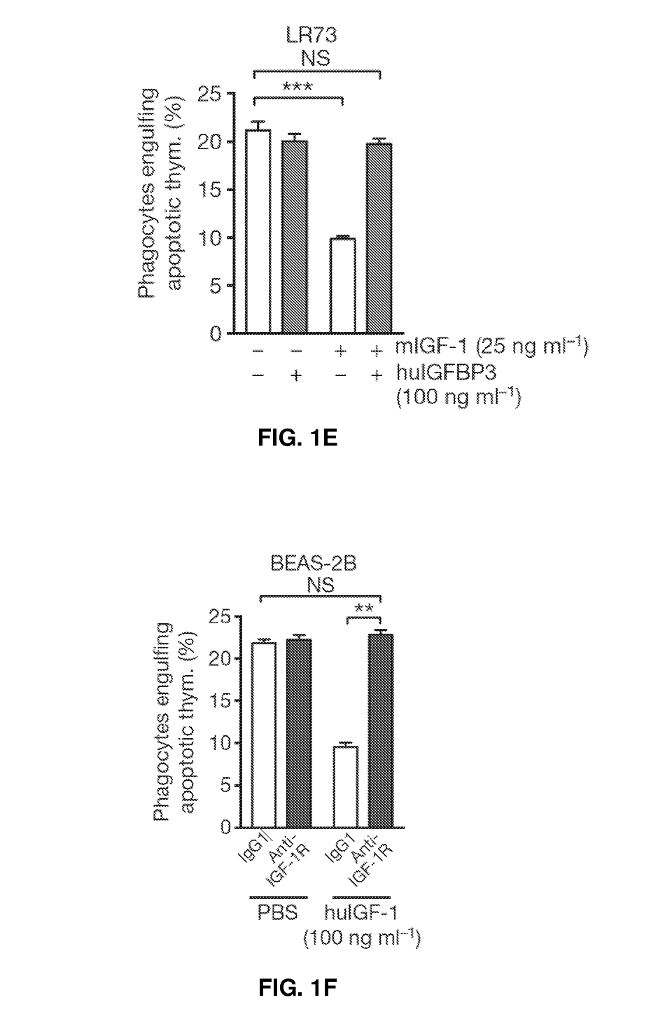

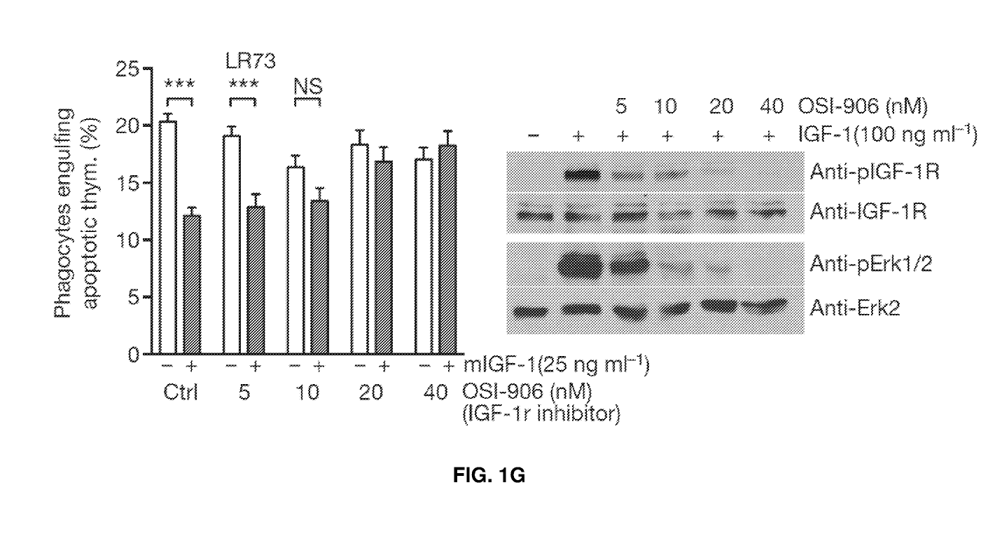

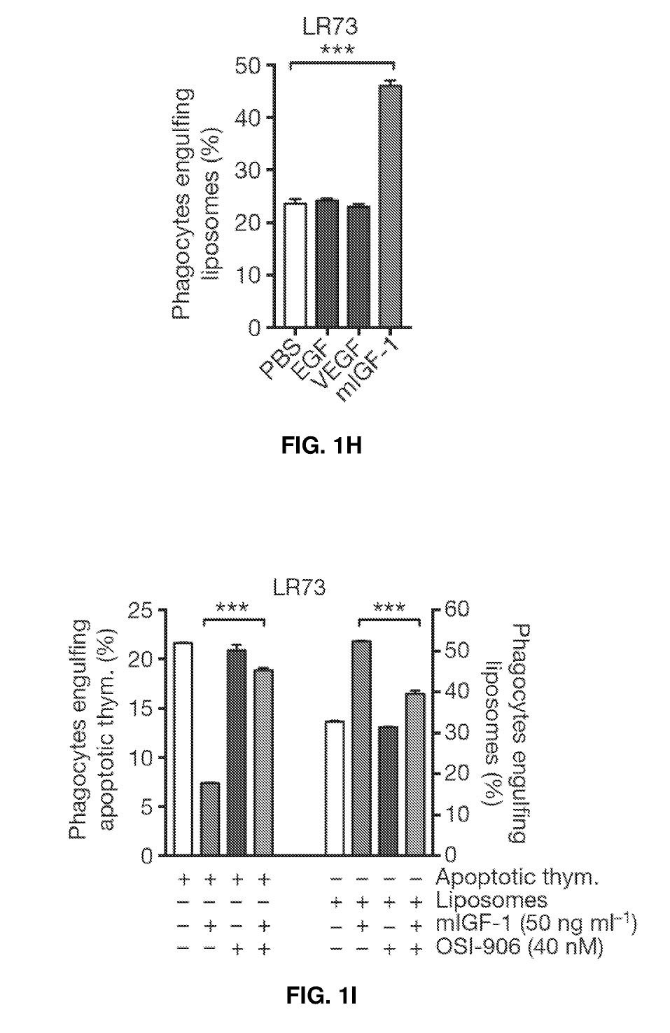

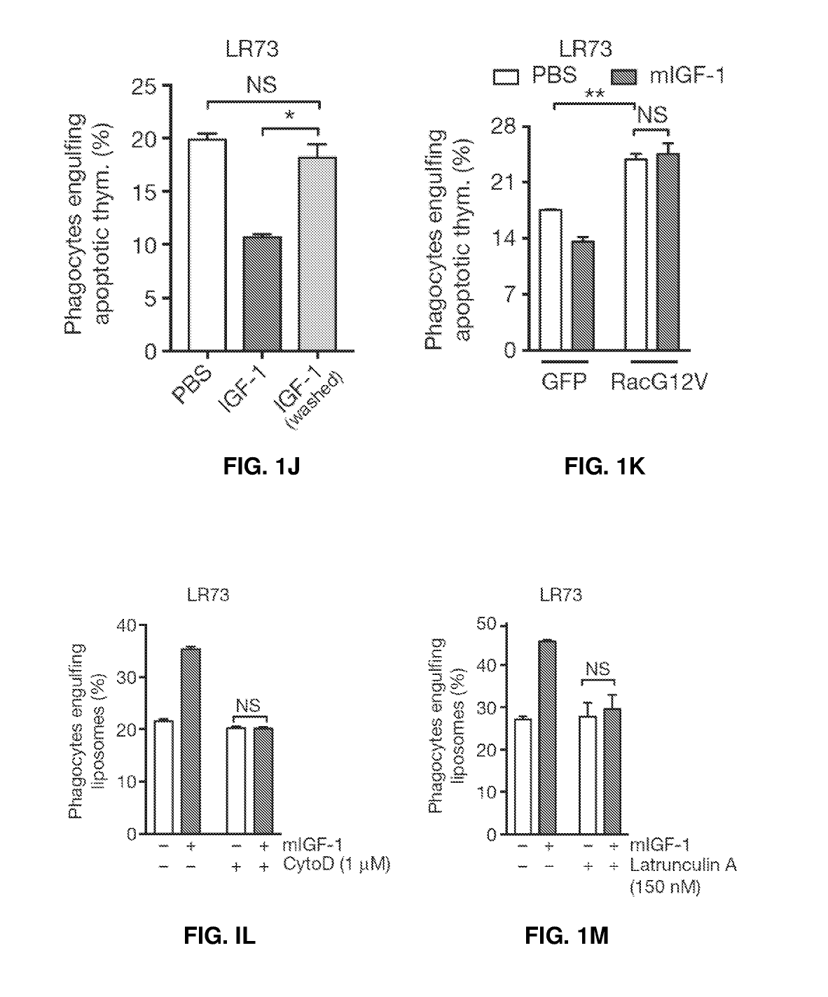

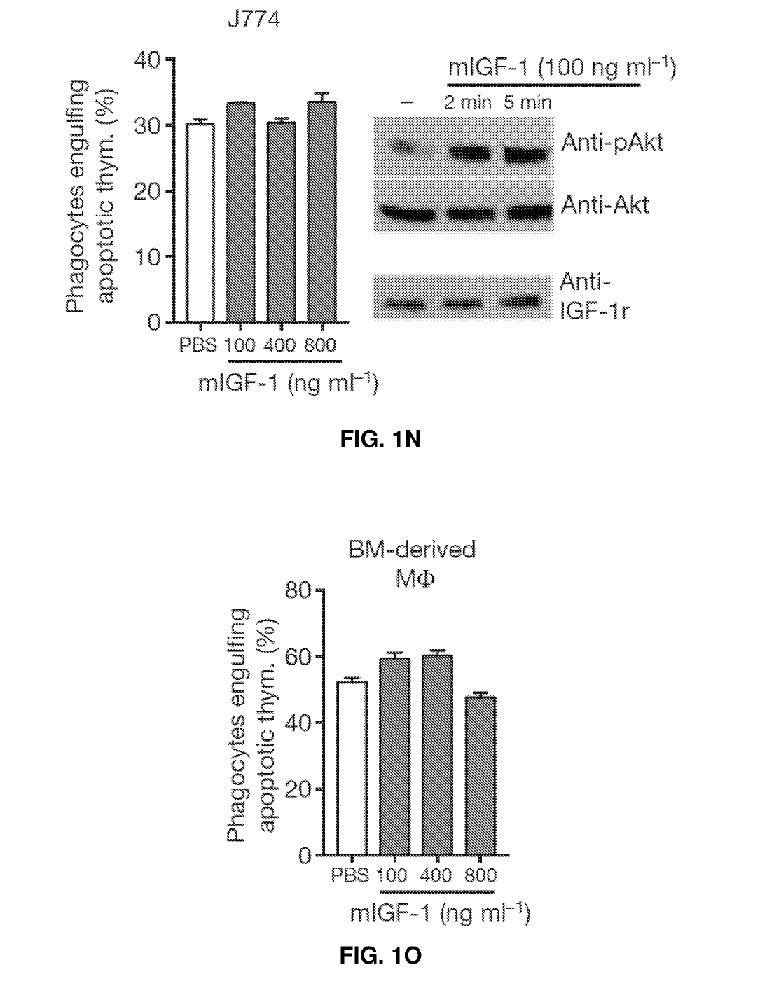

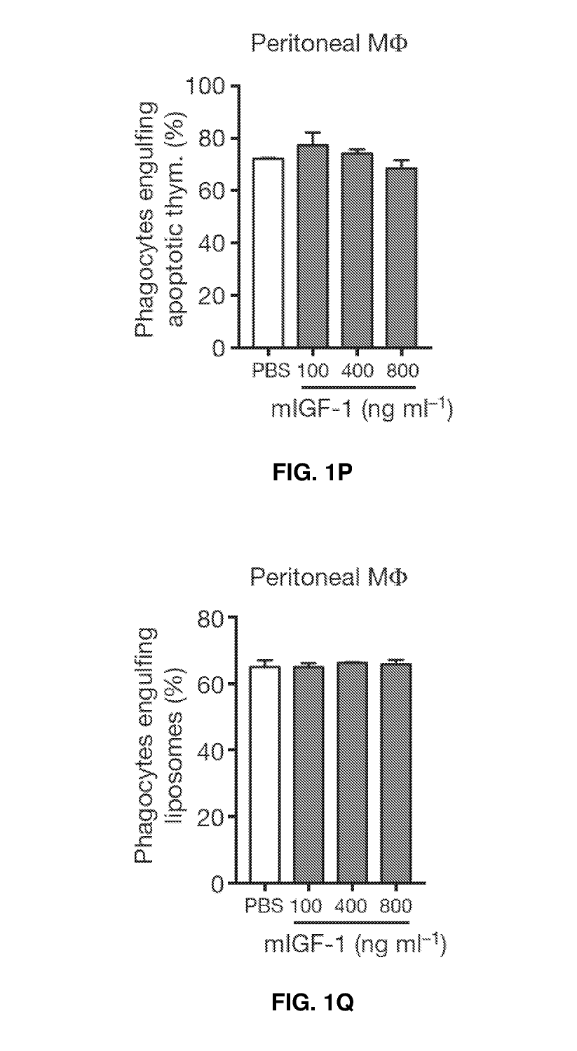

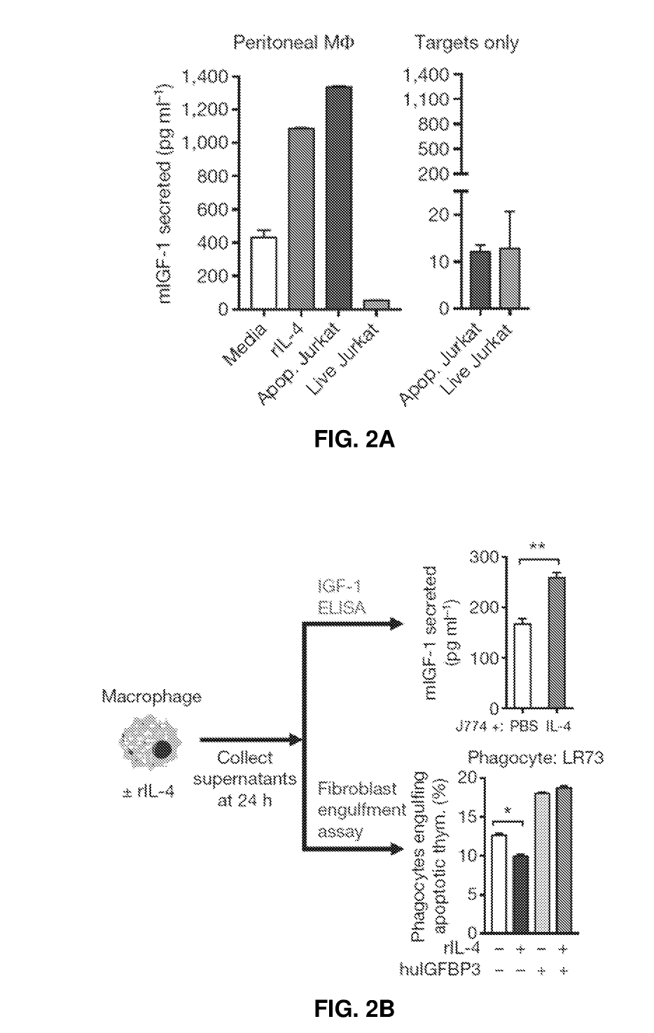

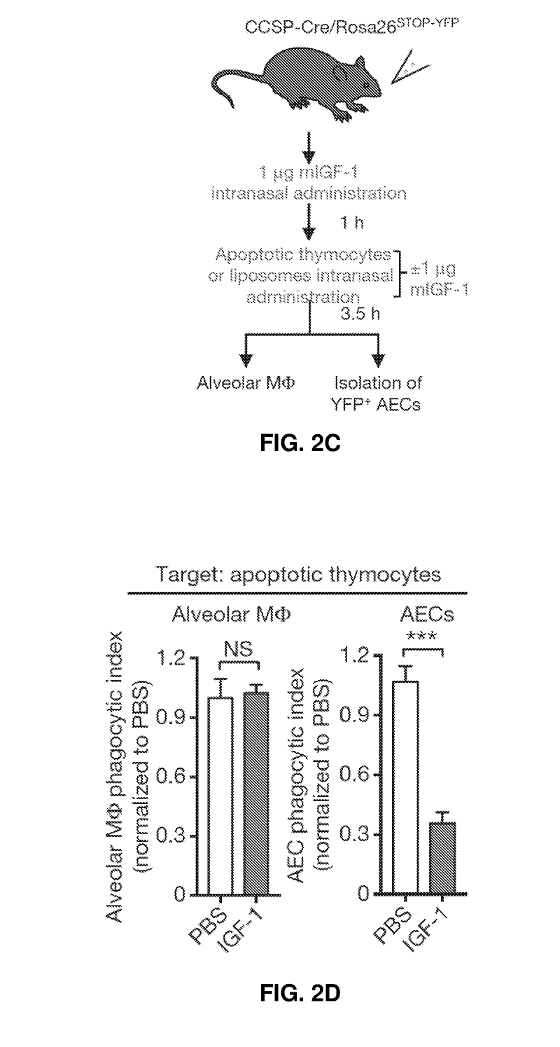

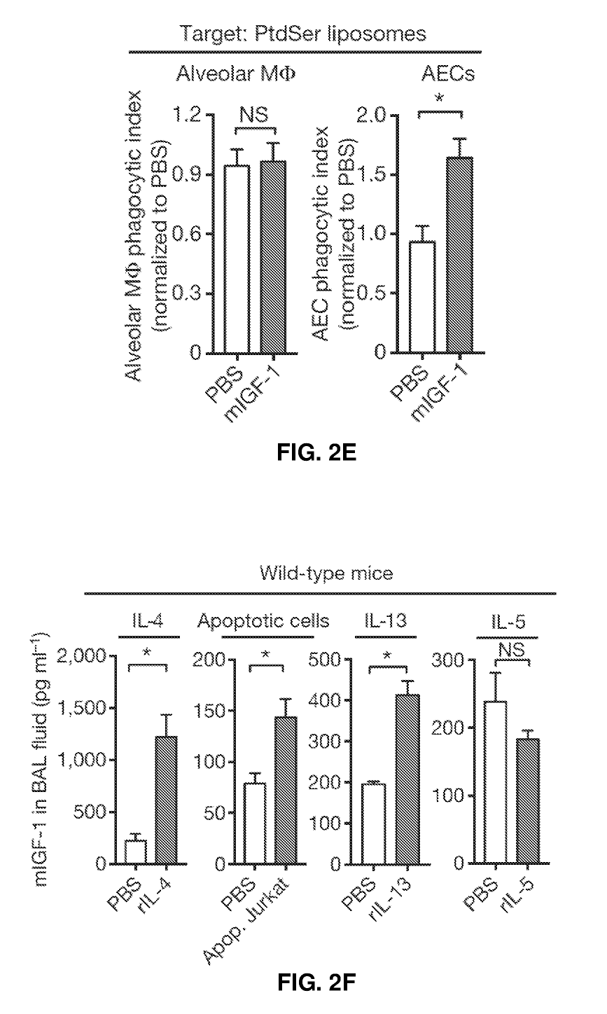

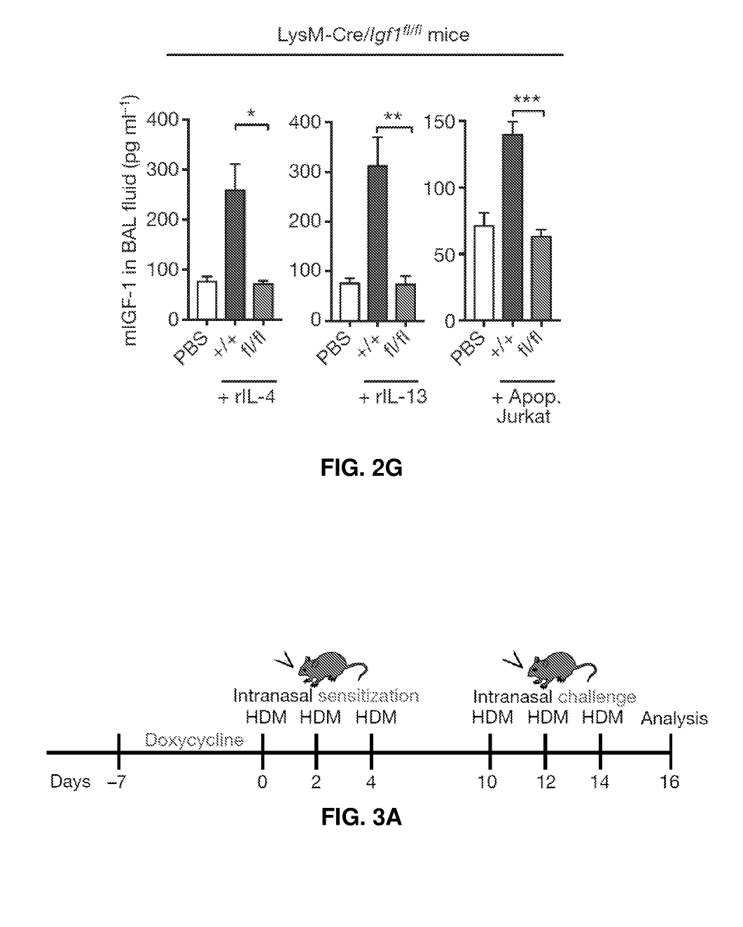

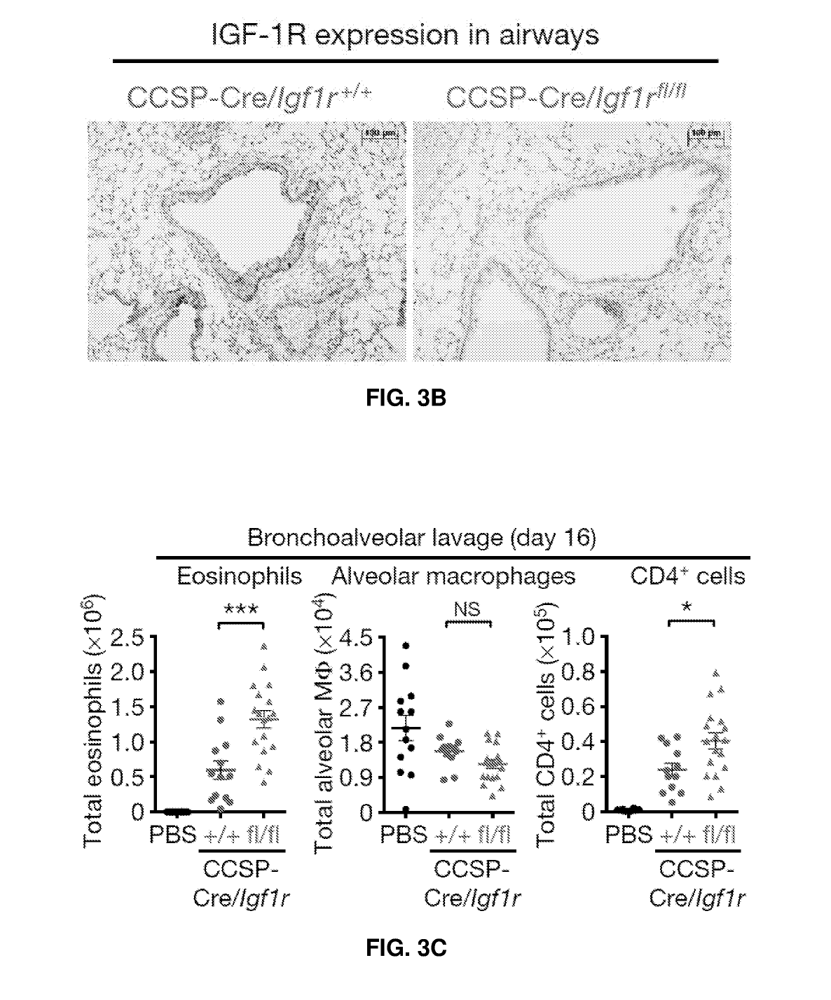

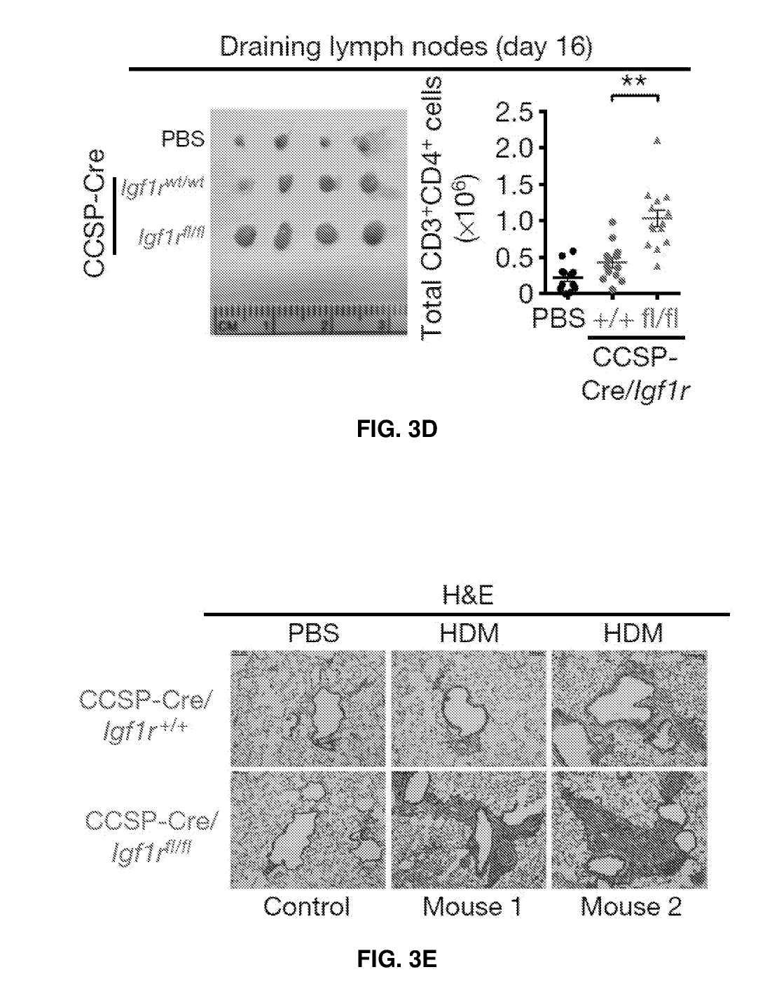

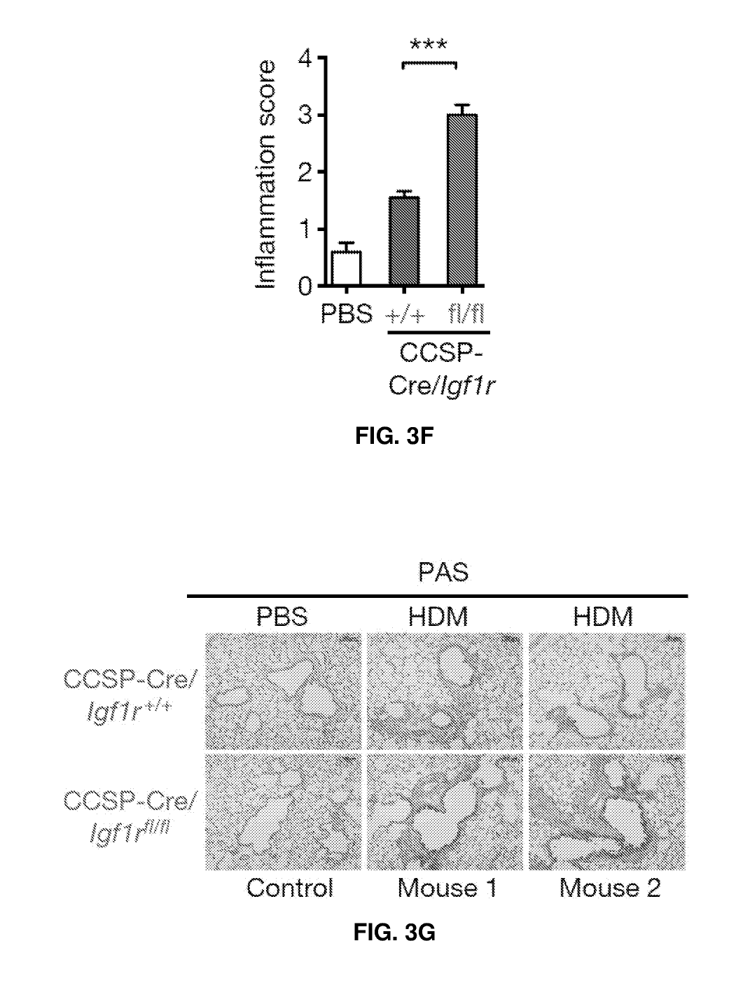

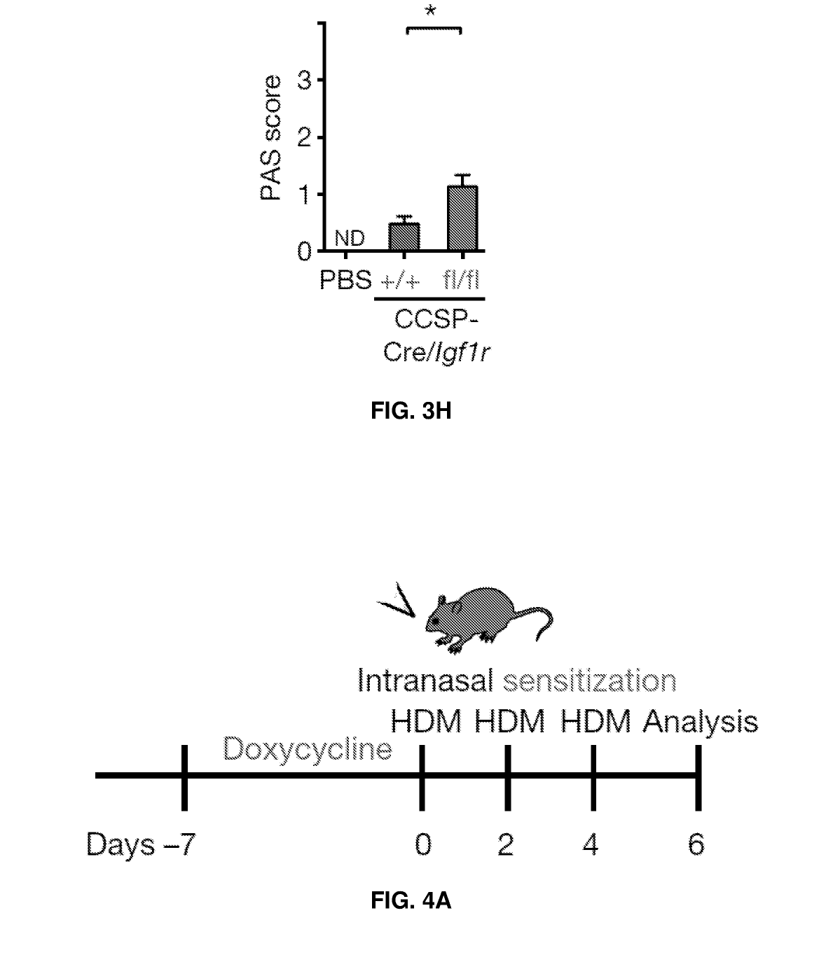

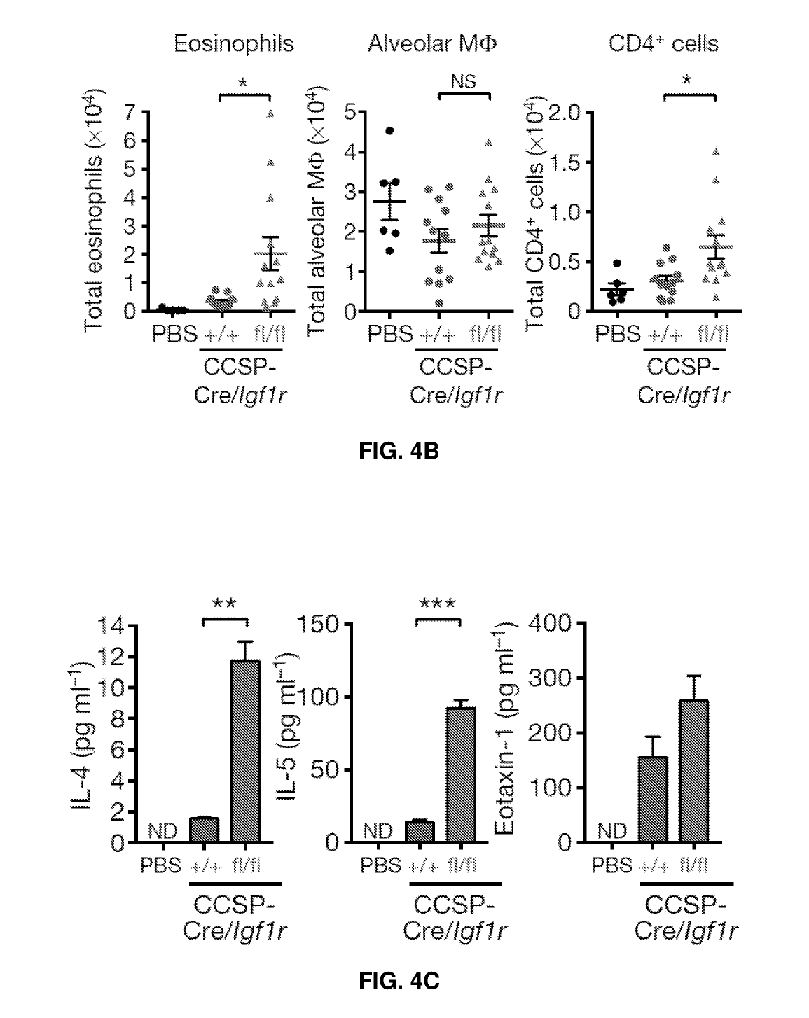

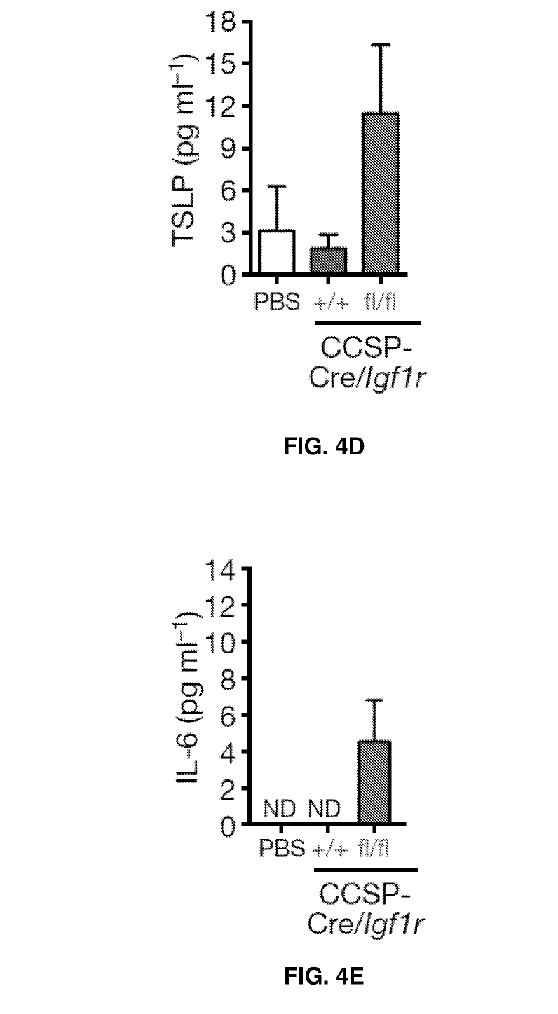

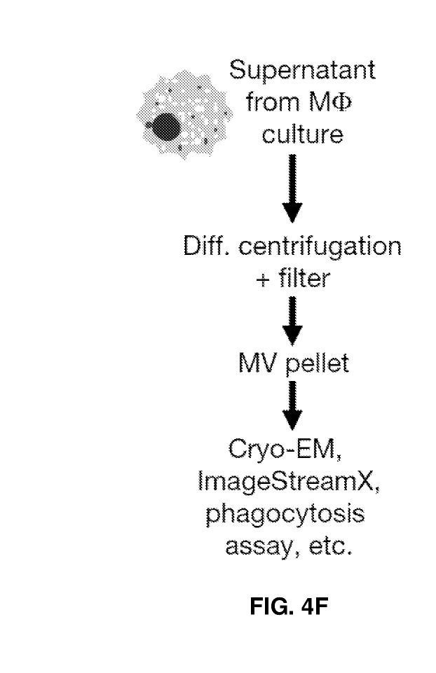

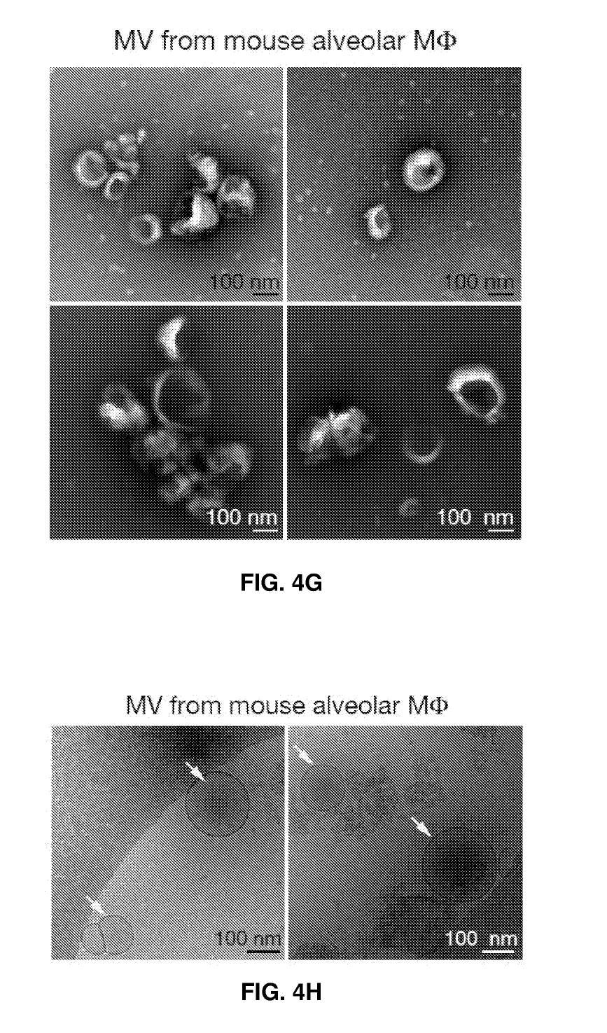

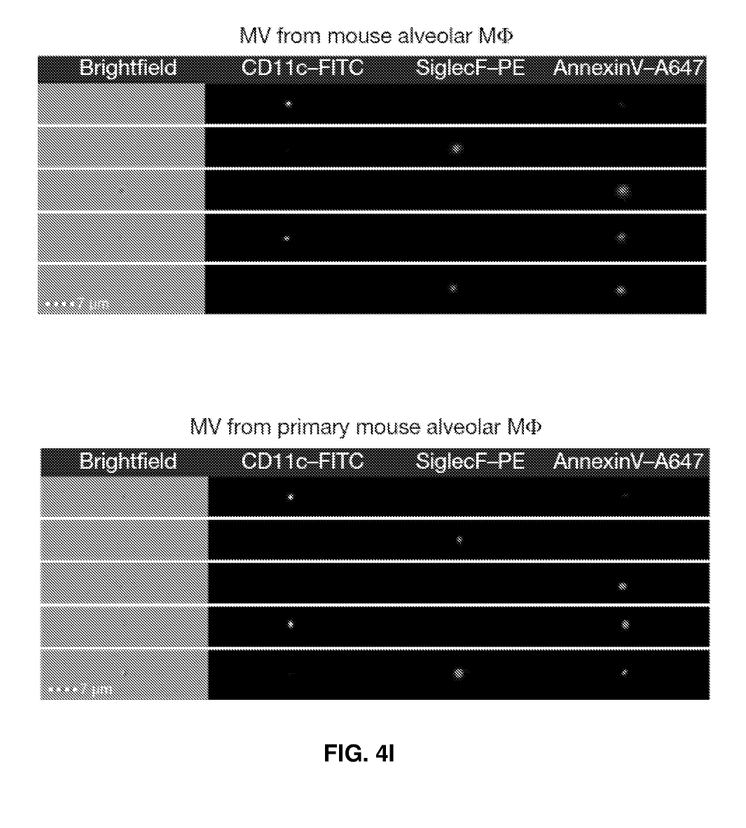

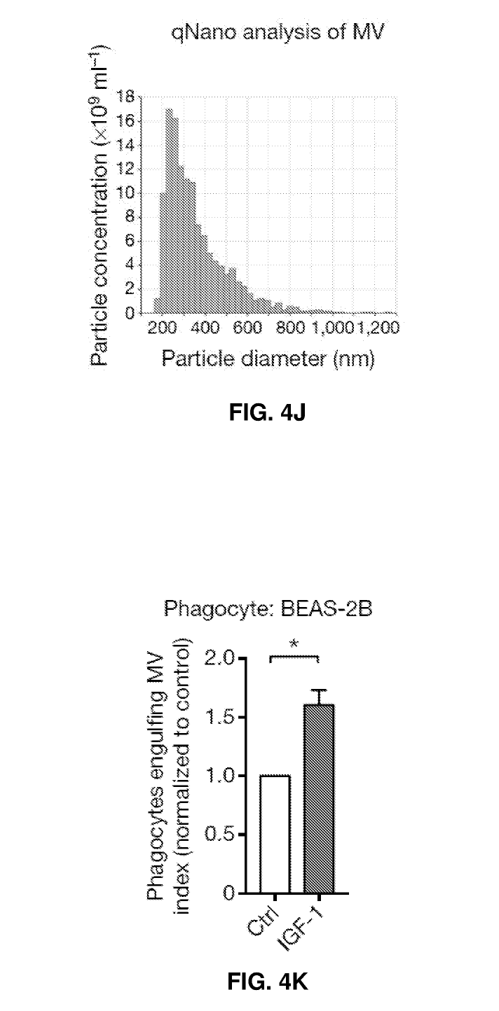

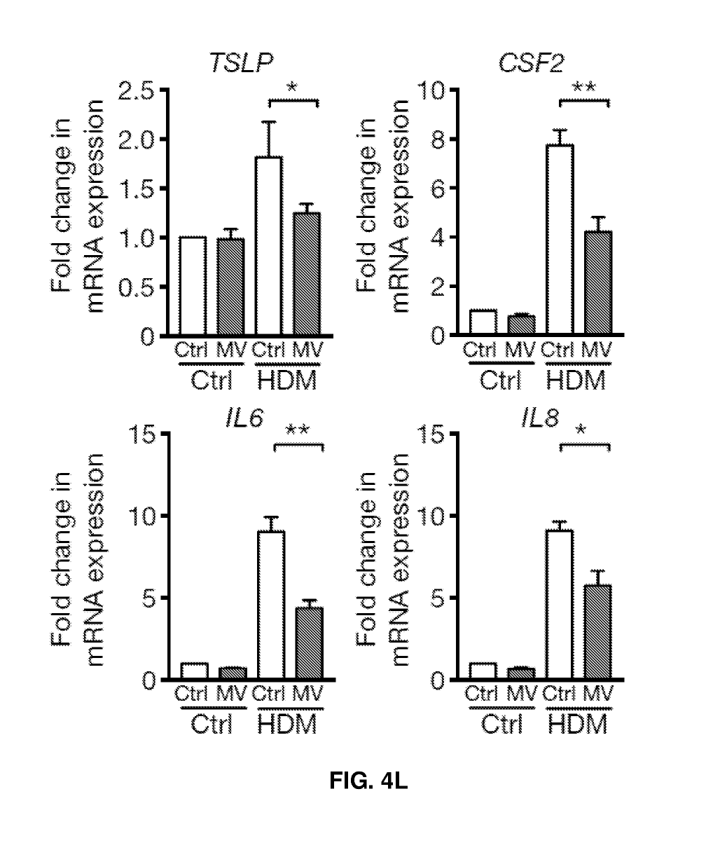

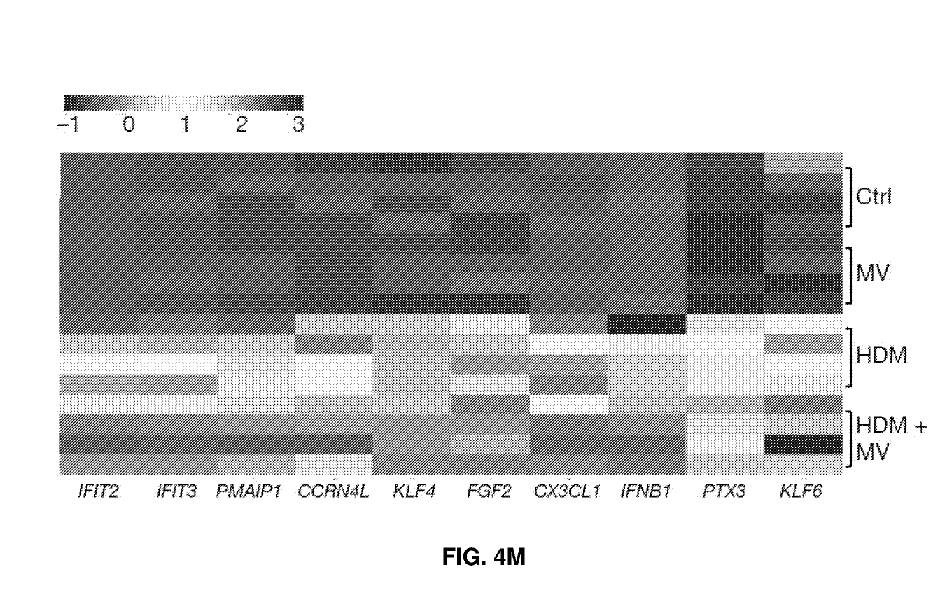

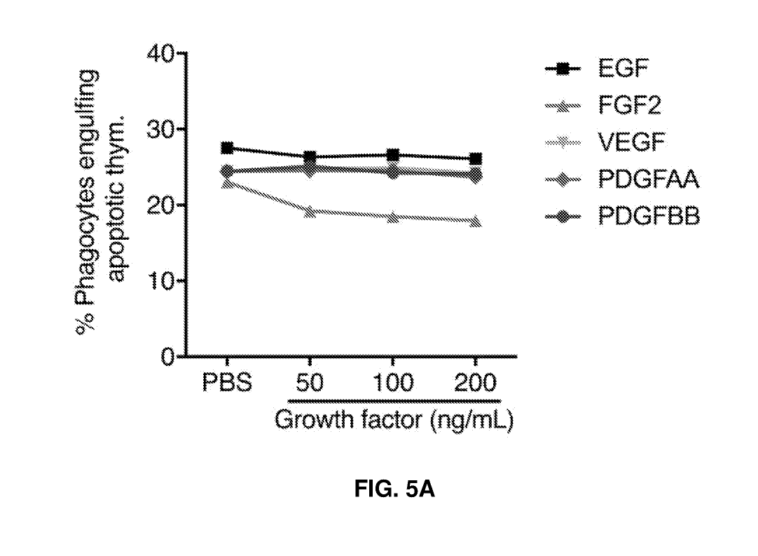

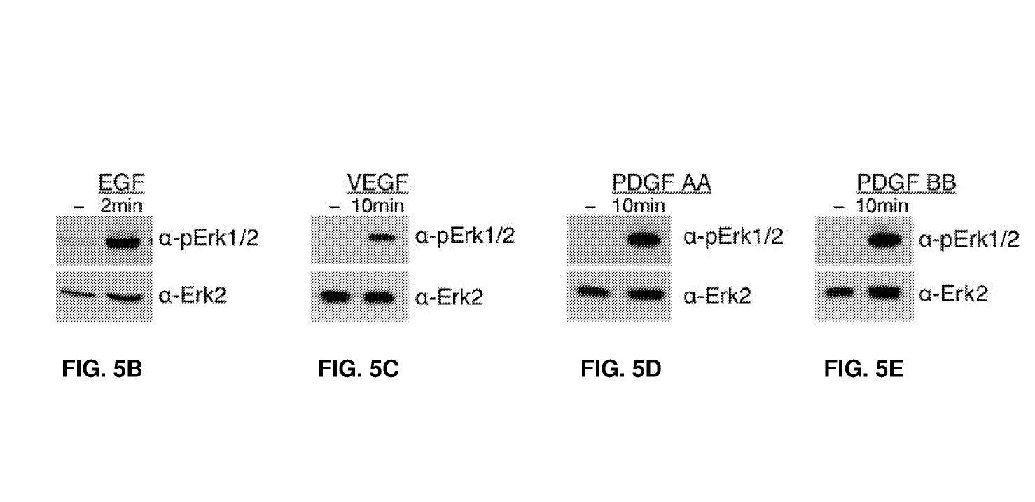

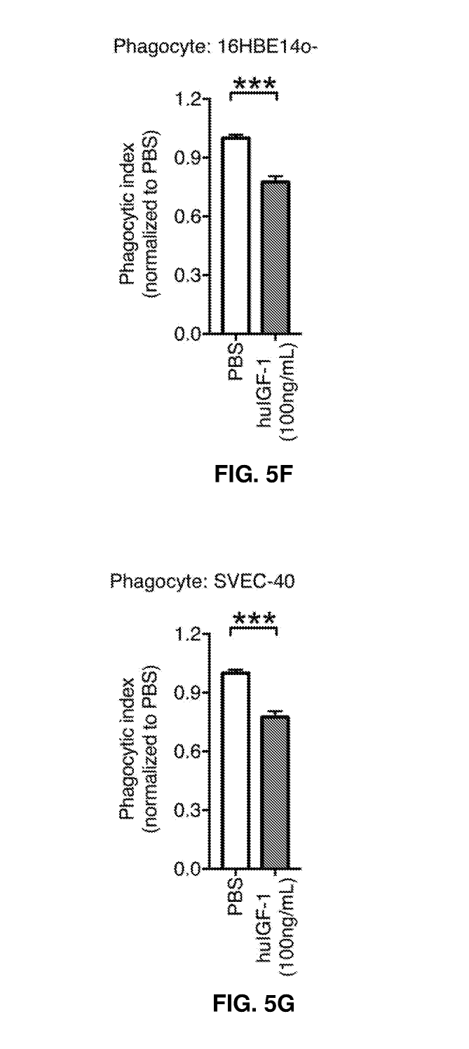

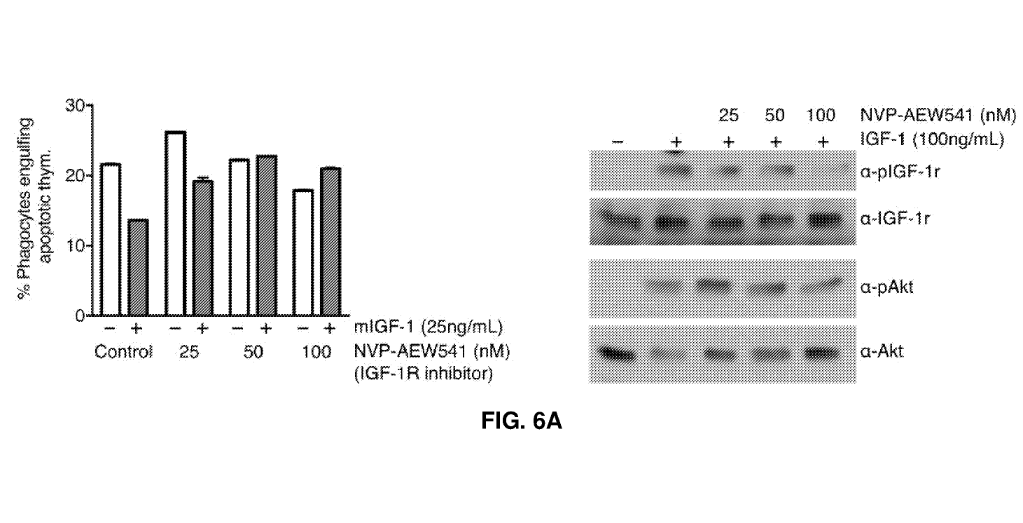

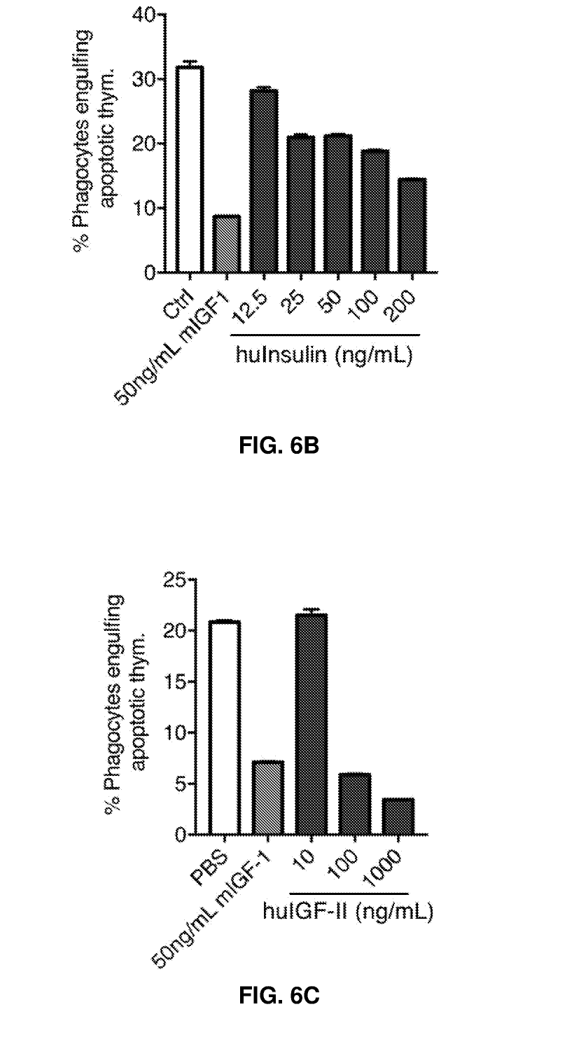

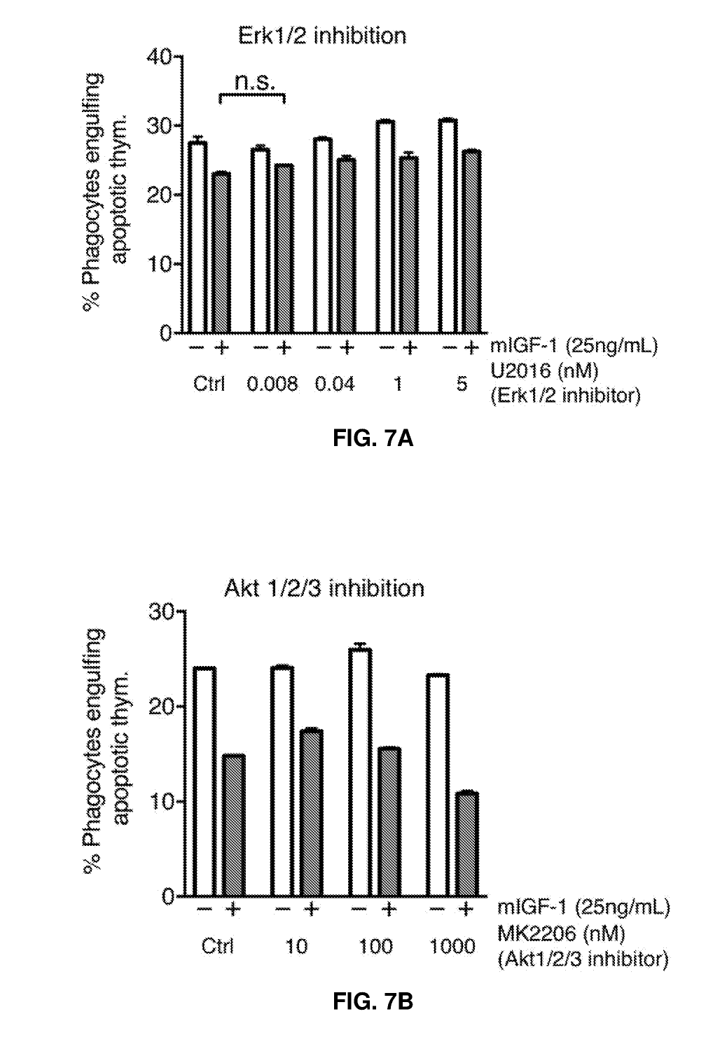

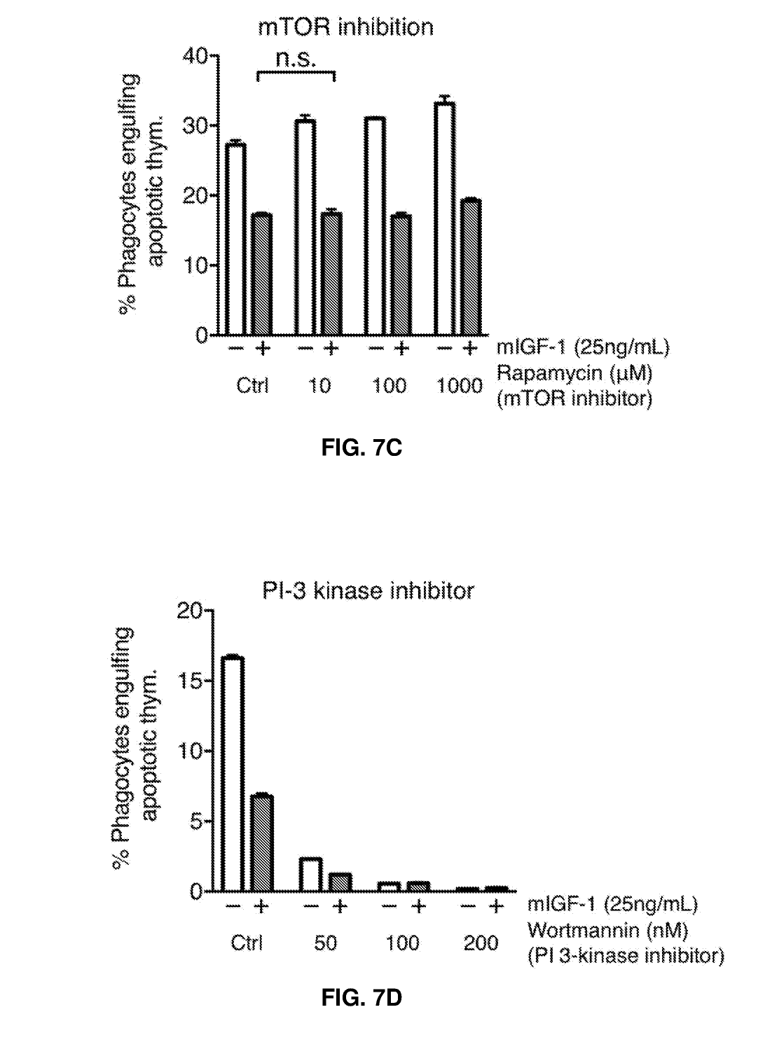

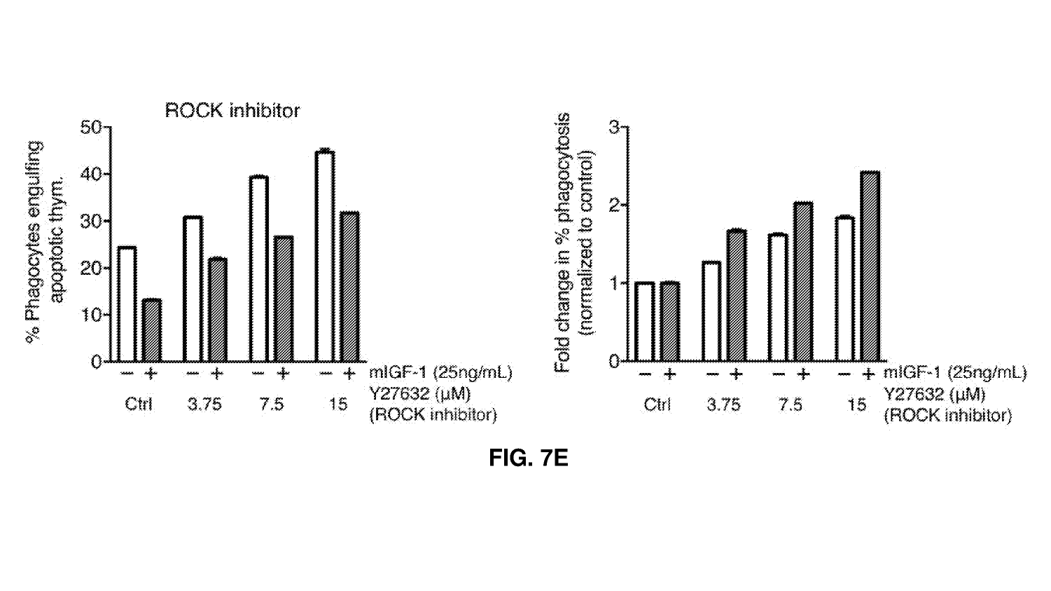

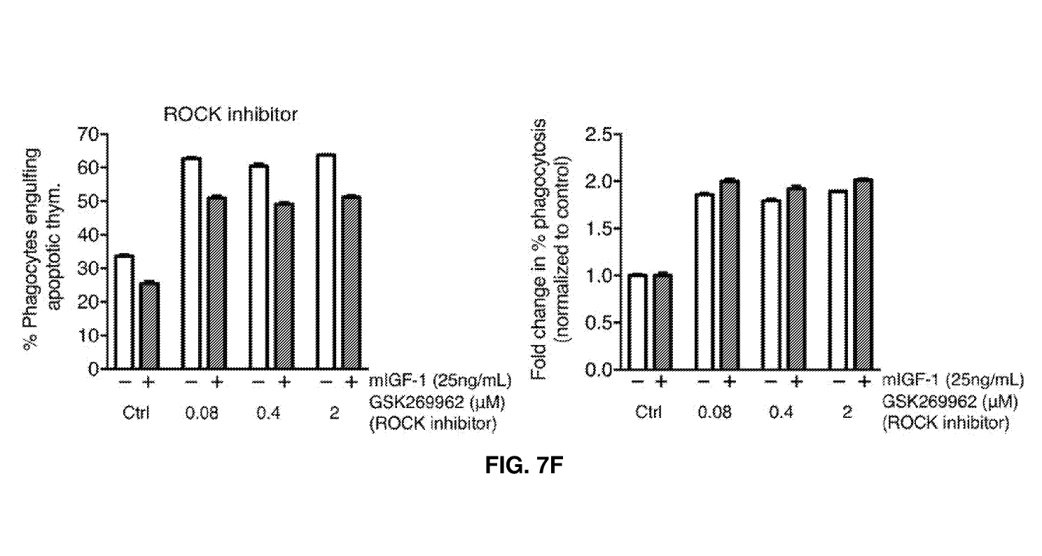

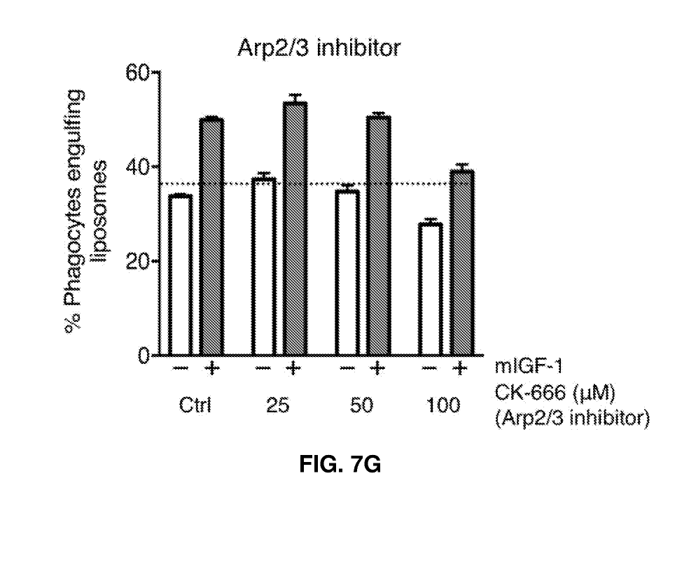

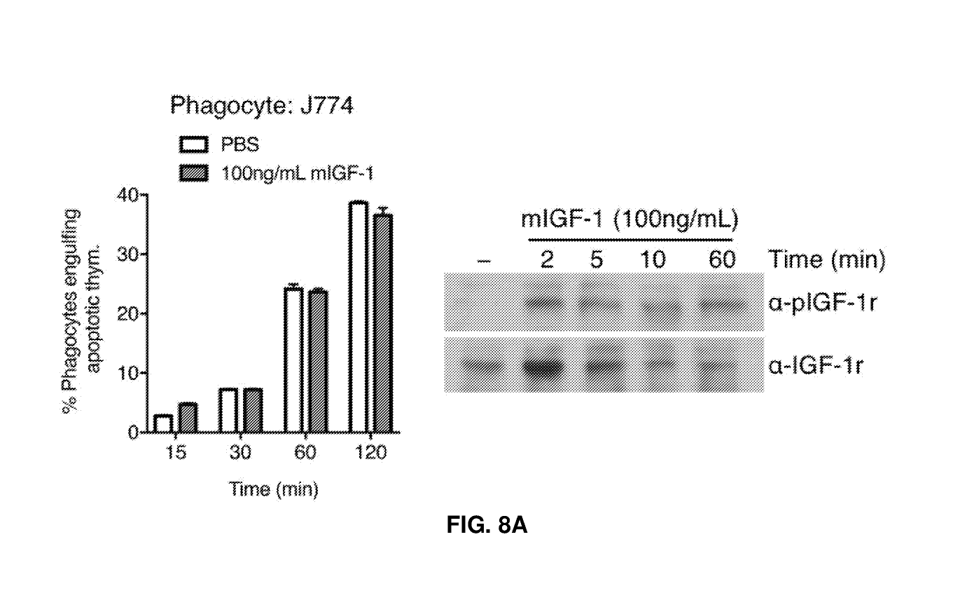

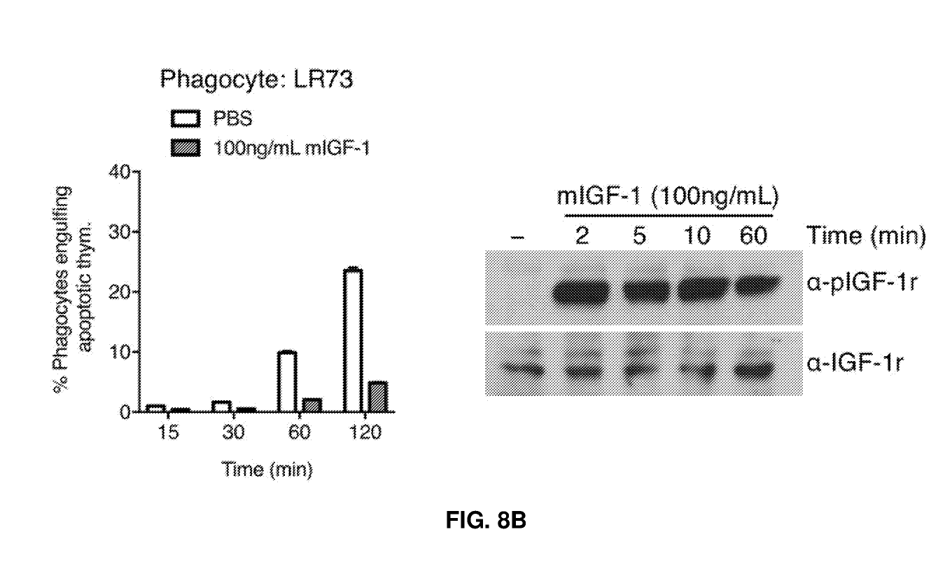

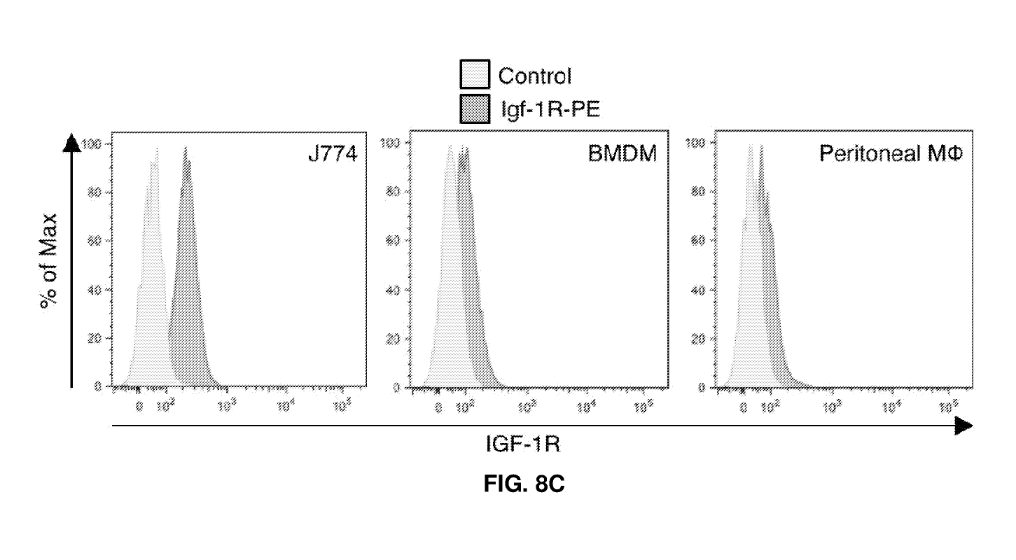

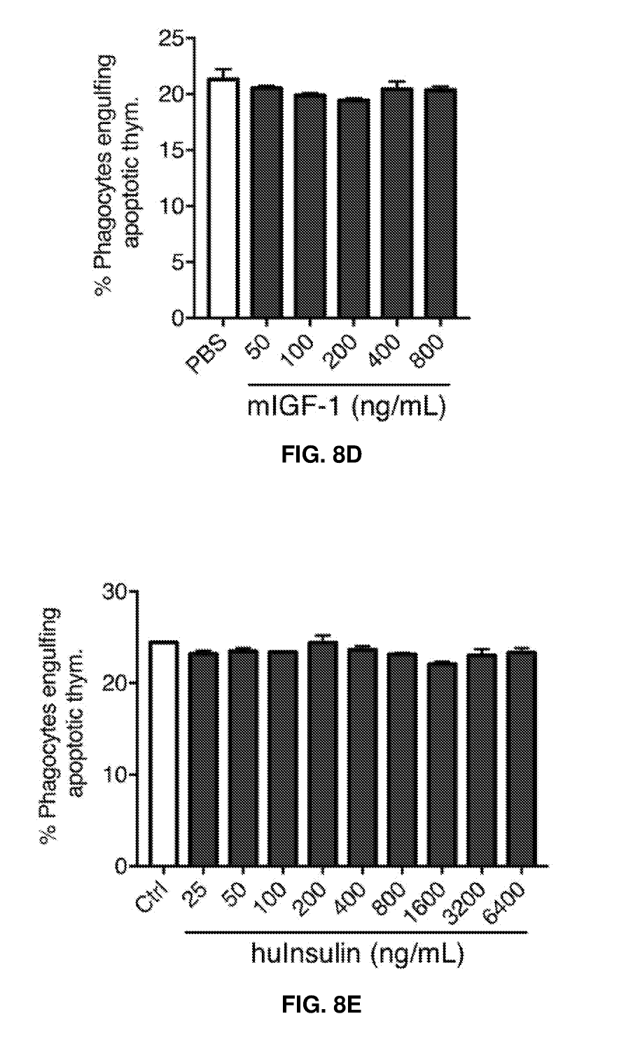

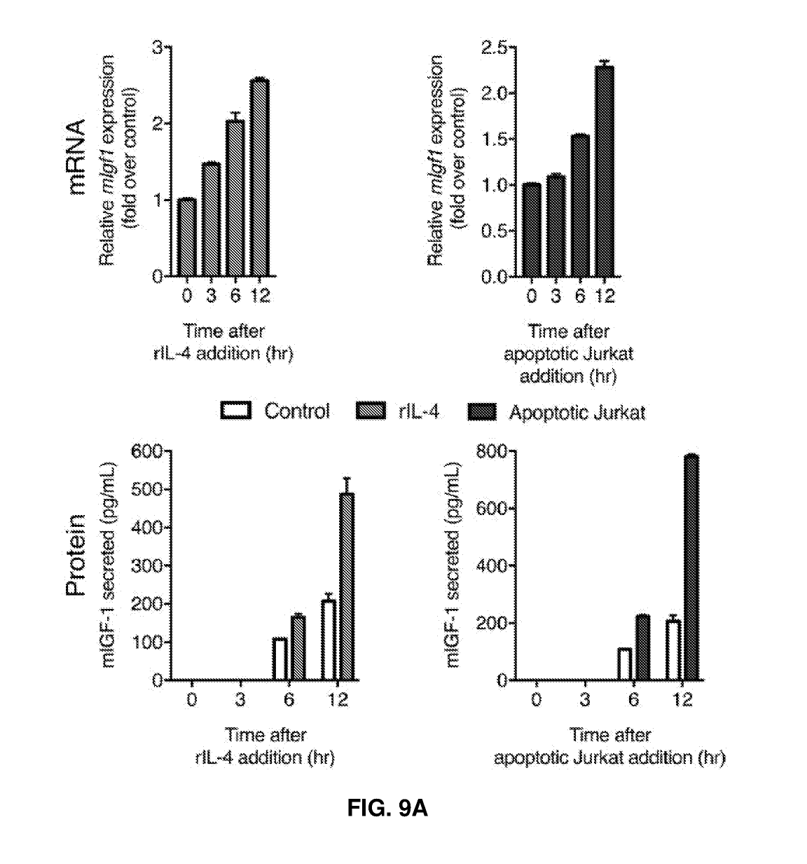

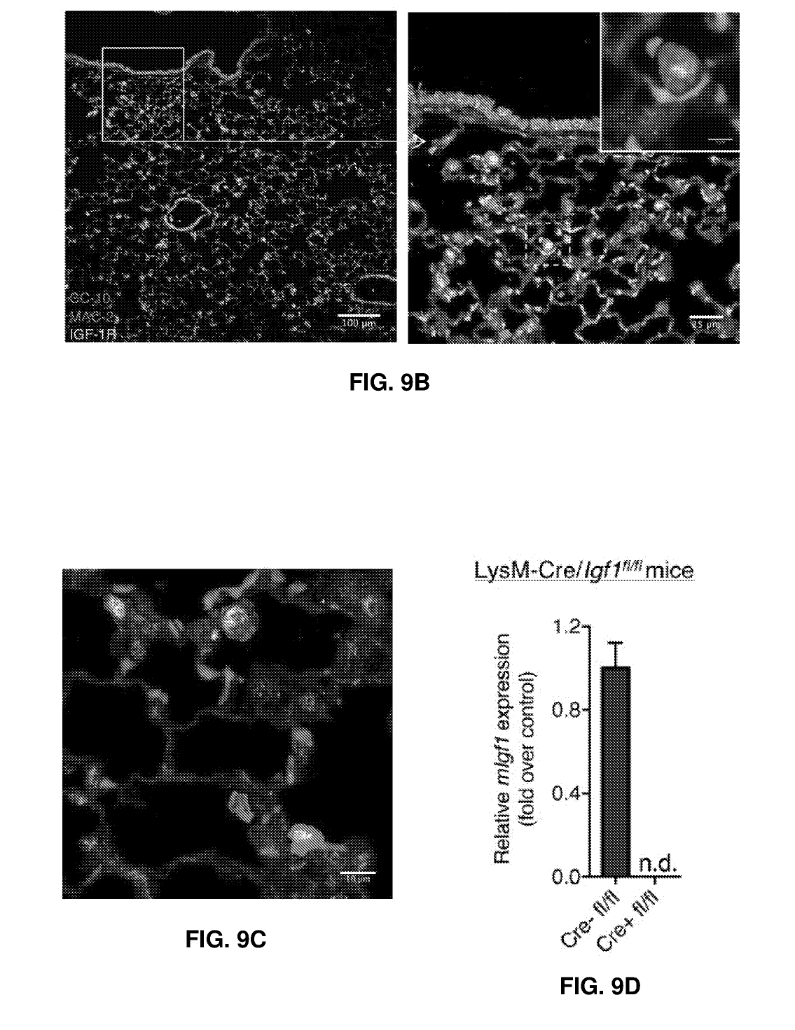

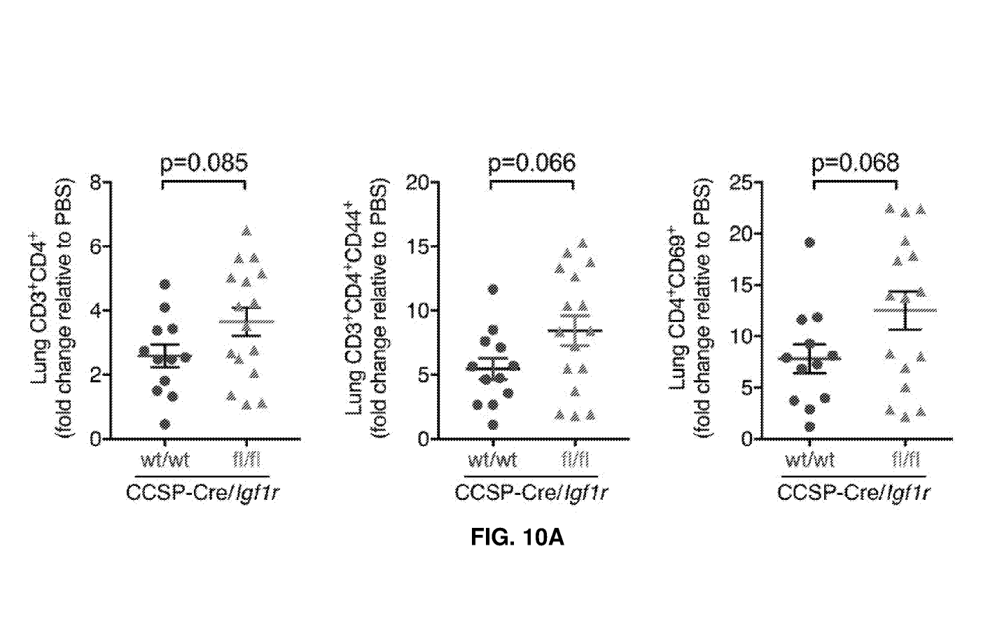

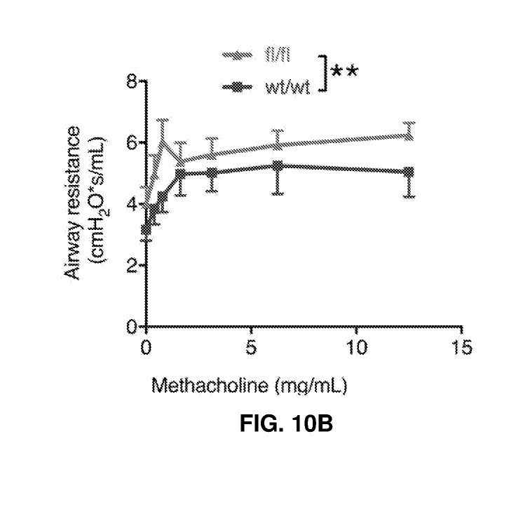

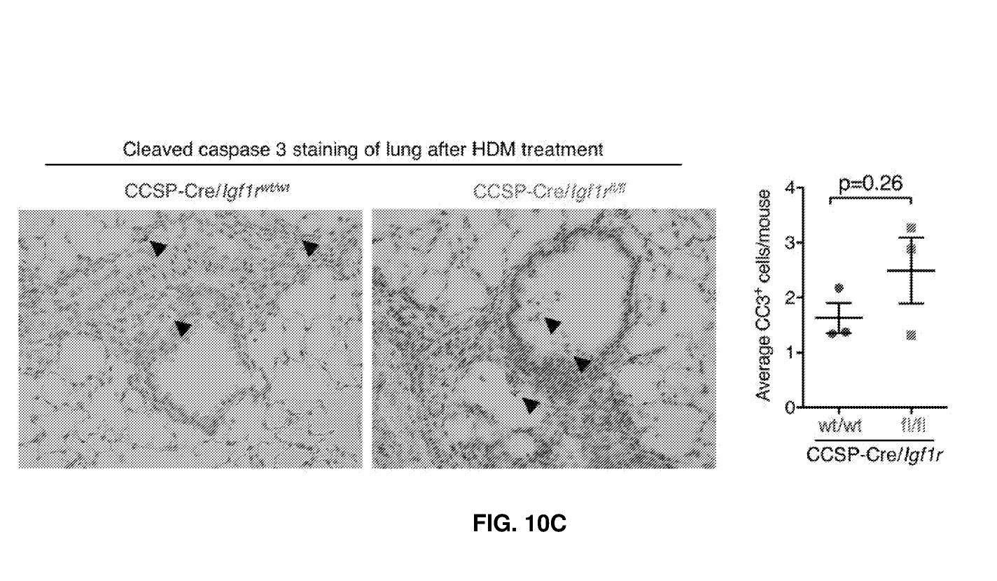

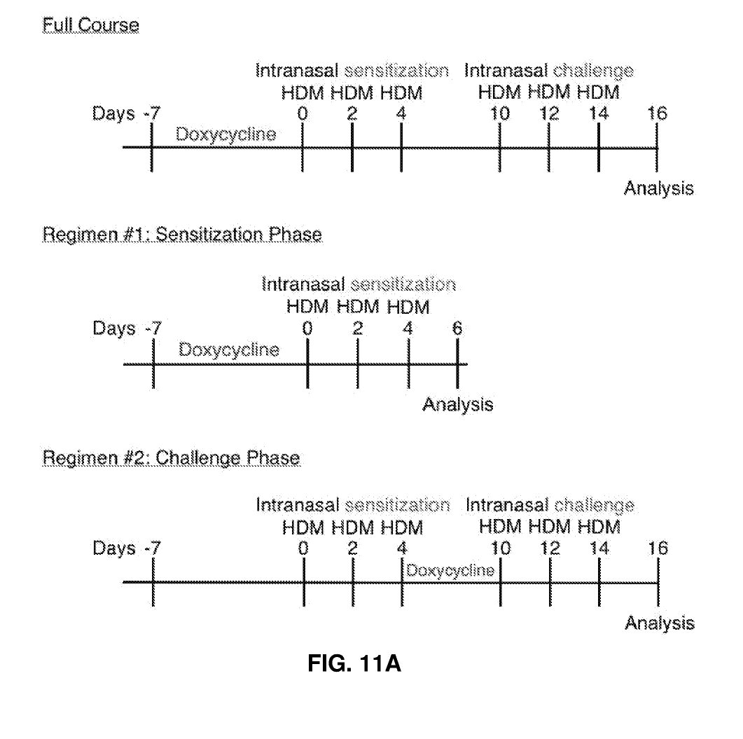

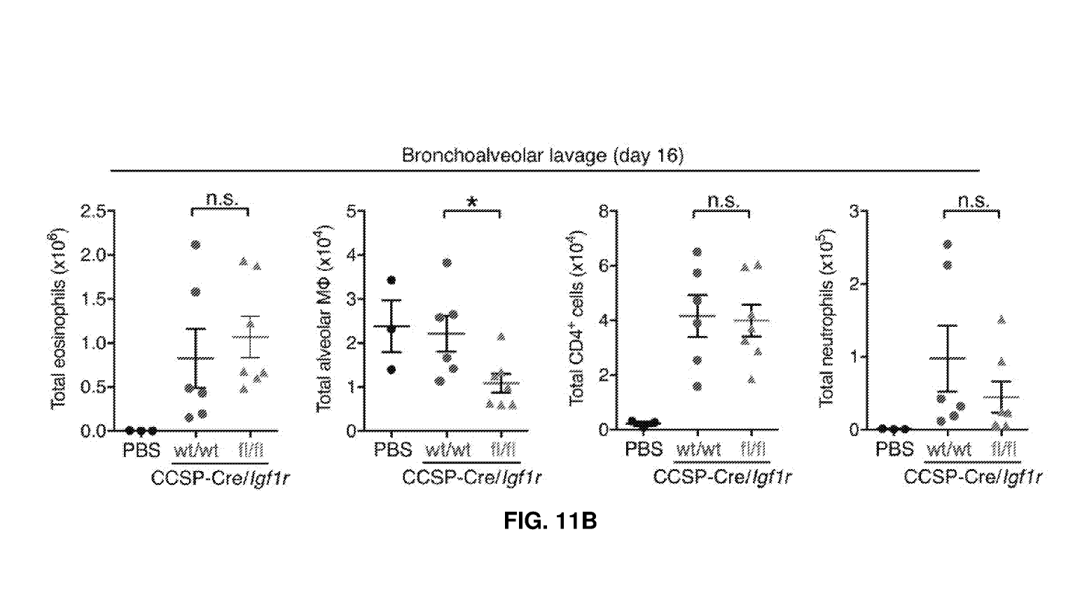

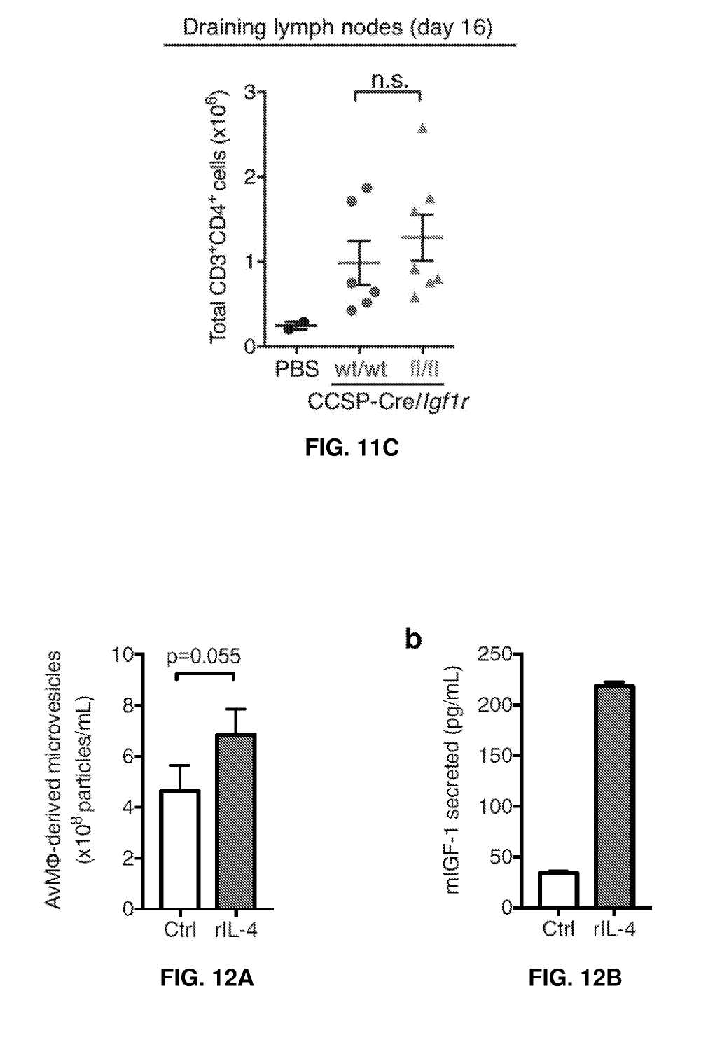

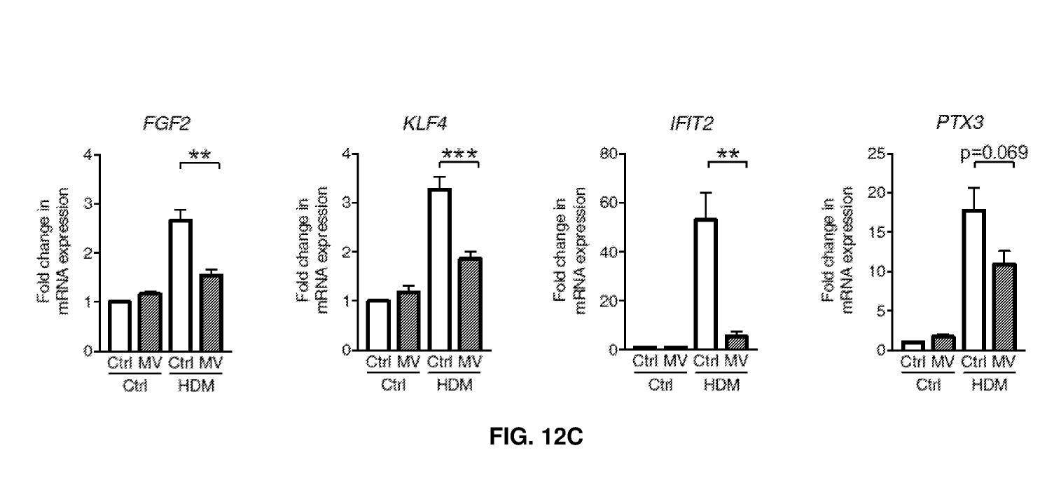

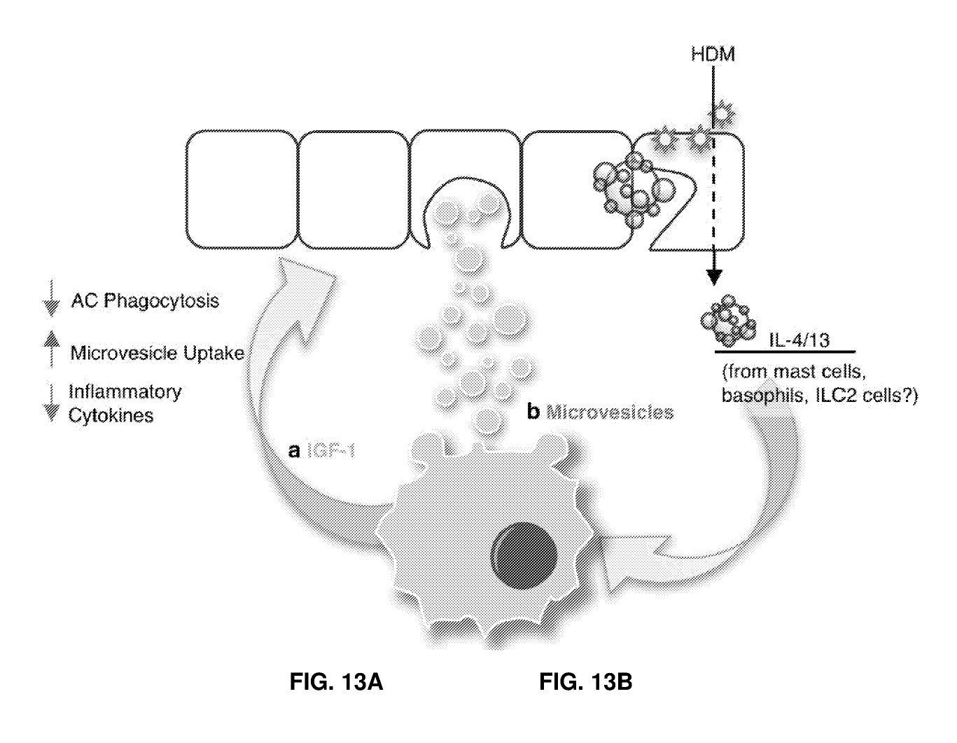

a, Effect of 11 growth factors on apoptotic cell engulfment by LR73 cells (each normalized to vehicle control). b, (Left) Engulfment by LR73 cells treated with increasing concentrations of IGF-I. (Right) Immunoblotting for IGF-1R and Akt phosphorylation. c, Apoptotic cell uptake by BEAS-2B cells treated with human IGF-I. d, IGF-I does not affect Annexin V binding to apoptotic thymocytes (n=6). e, f, Reversal of IGF-I mediated engulfment inhibition of LR73 cells by IGBFP3 (e), or BEAS-2B epithelial cells by IGF-IR neutralizing antibody (f). g, IGF-I-mediated reduction in engulfment is reversed by the IGF-1R inhibitor OSI-906 (left), and immunoblotting for IGF-IR and Erk1/2 phosphorylation (right). h, Liposome uptake by LR73 cells treated with either IGF-1 (50 ng/mL), EGF or VEGF (100 ng/mL each). i, Enhanced liposome uptake by IGF-1 is reversed by OSI-906. j, LR73 cells pretreated with IGF-I were washed, and incubated with apoptotic thymocytes in the presence or absence of IGF-I. k, Engulfment by LR73 cells transfected with RacG12V treated and with mIGF-1. l, m, Engulfment by LR73 cells treated with Cytochalasin D (1) or Latrunculin A (m) and incubated with liposomes with or without IGF-1. n, (Left) Phagocytosis of apoptotic thymocytes by J774 macrophage cells treated with IGF-I. (Right) Immunoblotting for IGF-IR expression and Akt phosphorylation. 0, p, Apoptotic cell engulfment by bone marrow derived macrophages or resident peritoneal macrophages treated with IGF-I. q, Liposome uptake by resident peritoneal macrophages treated with IGF-I. For a-q, except d, n=3; representative experiment is shown and data are mean±s.d. n.s.—not significant. p-value of <0.05 (*), <0.01 (**), or <0.001 (***). See Supplementary FIG. 1 in Han et al., Nature 2016, for uncropped immunoblots.

FIG. 2, comprising FIGS. 2a-2g, demonstrates that Macrophages produce IGF-I during apoptotic cell clearance.

a, (Left) IGF-1 secretion by peritoneal macrophages stimulated with IL-4, apoptotic Jurkat cells or live Jurkat cells. (Right) Apoptotic or live Jurkat cells do not produce IGF-1 (representative of n=3), mean±s.d. b, J774 cells were treated with rIL-4. After 24 hrs, half of the supernatant was assessed for IGF-1 secretion by ELISA, (right panel) and the other half was incubated with PBS or IGBP3 and then tested in the phagocytosis using LR73 cells (right) (representative of n=3), mean±s.d. Note: addition of IGFBP3 increases the basal phagocytosis due to low basal levels of IGF-1 found in the supernatant of unstimulated J774 cells. c, d, e, CCSP-Cre mice with YFP⁺ Club cells were pre-treated with 1 μg IGF-1 intranasally for 1 hr and then administered targets with or without IGF-1. Uptake of apoptotic thymocytes (d, n=6 per group) or liposomes (e, n=3 per group) by alveolar macrophages and lung epithelial cells were then assessed. f, Wild-type mice were given PBS, IL-4, apoptotic cells, IL-5, or IL-13 intranasally for 2 consecutive days, and the BAL fluid assessed for IGF-1 (n=2-3 mice per group for cytokines, n=5, 8 for apoptotic cell instillation). g, LysM-Cre/Igf1r^{wt/wt} or Igf1r^{fl/fl} mice were given PBS, IL-4 or IL-13, or apoptotic cells intranasally, and BAL fluid assessed for IGF-1 (n=6, 6, 4 mice per group for rIL-4; n=6, 4, 4 for rIL-13; n=6, 9, 9 mice per group for apoptotic cell instillation). Data are mean±s.e.m unless otherwise indicated.

FIG. 3, comprising FIGS. 3a-3h, demonstrates that Mice lacking IGF-1R in airway epithelial cells have exacerbated airway inflammation.

a, Schematic of HDM induced allergic airway inflammation. b, Representative images showing IGF-1R expression in bronchial epithelial cells, and its loss in CCSP-_{ntTA/tet( )}-Cre/Igf1r^{wt/wt} mice treated with doxycycline. c, Numbers of eosinophils, alveolar macrophages, and CD4⁺ T-cells in the BAL fluid of CCSP-Cre/Igf1r^{wt/wt} and Igf1r^{fl/fl} mice administered PBS or HDM (each dot represents a mouse). d, (Left) Representative lung draining lymph nodes from CCSP-Cre/Igf1r^{wt/wt} and CCSP-Crea/Igf1r^{fl/fl} mice that were given PBS or HDM. (Right) Total CD4⁺ T cell counts from lymph nodes. e, f, g, h, Representative hematoxylin and eosin (H&E) images (e) or PAS staining (g) of lung sections from CCSP-Cre/Igf1r^{wt/wt} and CCSP-Cre/Igf1r^{fl/fl} mice given PBS or HDM (n=3-4 mice per condition). Representative histological scoring of inflammation (f) and PAS staining (h) (n=6-10 sections and 3 mice per condition). All data are presented as mean±s.e.m.

FIG. 4, comprising FIGS. 4a-4h, demonstrates that IGF-1R expression in airway epithelial cells is required in the sensitization phase of airway inflammation.

a, Schematic of IGF-1R deletion prior to the sensitization phase to assess its effect on early stages of inflammation. b, Numbers of eosinophils, alveolar macrophages, and CD4⁺ T-cells in the BAL of CCSP-Cre/Igf1r^{wt/wt} and CCSP-Cre/Igf1r^{fl/fl} mice primed with PBS or HDM. c, d, e, Analysis of IL-4, IL-5, eotaxin-1, and IL-6 (via Luminex c, e, n=3 mice per group) and TSLP (by ELISA, d, n=2, 7, 9 mice per group) in the BAL fluid from representative CCSP-Cre/Igf1r^{wt/wt} and CCSP-Cre/Igf1r^{fl/fl} mice primed with PBS or HDM. f, Schematic of generation and isolation of alveolar macrophage derived microvesicles. g, h, Representative negative-stain EM (g) or cryo-EM (h) images of microvesicles isolated from mouse alveolar macrophages. Images show spherical membrane-bound structures of a range of sizes (yellow arrows). i, Representative ImageStream™ images of microvesicles isolated from mouse alveolar macrophage cell line and primary mouse alveolar macrophages and stained for alveolar macrophage markers. j, Tunable resistive pulse sensing analysis of microvesicles from alveolar macrophages using qNano, pore size 400 nm, to determine frequency and sizing of microvesicles (representative of n=3). k, BEAS-2B cells treated with IGF-1 (100 ng/mL) were assessed for uptake of alveolar macrophage derived microvesicles (MV) (n=4). 1, BEAS-2B cells were treated with HDM either in the presence or absence of alveolar macrophage derived microvesicles (MV) for 3 hours and assessed for expression of TSLP, CSF2, IL6, IL8 (n=4). m, Heatmap of top 10 differentially expressed genes from RNA-seq analysis of BEAS-2B cells exposed to HDM with or without alveolar macrophage-derived microvesicles. Data presented as mean±s.e.m. n.d. is not detected

FIG. 5, comprising FIGS. 5a-5g, demonstrates that IGF-1, but not EGF, VEGF, PDGF AA/BB, suppresses phagocytosis of apoptotic cells in non-professional phagocytes.

a, Representative engulfment assay in which LR73 cells were treated with indicated growth factors at increasing concentrations and assessed for engulfment of apoptotic thymocytes (n=3). b-e, Serum-starved LR73 cells were stimulated with 100 ng/mL of specified growth factors for indicated time and the phosphorylation of Erk1/2 was determined by immunoblotting (n=2). f, g, Representative engulfment assay in which the uptake of apoptotic thymocytes by 16HBE14o-human airway epithelial cells (b) or SVEC-40 endothelial cells (c) is dampened by IGF-1 treatment (n=3). Error bars represent s.d.

FIG. 6, comprising FIGS. 6a-6d, demonstrates that Insulin and IGF-II also decrease apoptotic cell engulfment, similar to IGF-1 that is reversed by treatment with NVP-AEW541.

a, (Left) Engulfment of apoptotic thymocytes by LR73 cells treated with various doses of NVP-AEW541, a small molecule inhibitor of IGF-1r (n=3). (Right) Representative immunoblot of LR73 cells stimulated with IGF-1 and treated with increasing doses of NVP-AEW541 (n=2). b, c, Engulfment of apoptotic thymocytes by LR73 cells treated with the indicated concentrations of human insulin (c) and human IGF-II (d) (n=2). Error bars represent s.d.

FIG. 3, comprising FIGS. 7a-7g, demonstrates that Blocking canonical signaling intermediates downstream of IGF-1 receptor signaling, Rho-kinase (ROCK), or Arp2/3 mediated functions, does not reverse the IGF-1 mediated engulfment modulation.

a-f, Engulfment of apoptotic thymocytes by LR73 cells treated with U2016 (Erk1/2 inhibitor) (a), MK-2206 (Akt1/2/3 inhibitor) (b), Rapamycin (mTOR inhibitor) (c), or Wortmannin (PI 3-Kinase inhibitor) (d), Rho kinase inhibitors Y27632 (e) or GSK269962 (f) in the presence or absence of IGF-1 (n=2-3). Initially, it appeared that inhibition of ROCK was able to partially rescue IGF-1 induced engulfment suppression. However, as ROCK inhibition basally increases phagocytosis of apoptotic cells (consistent with what has been previously reported), we normalized the change in phagocytosis for each inhibitor concentration to the appropriate control (right panel). After normalizing, we observed that ROCK inhibition did not increase corpse uptake in LR73 cells in the presence of IGF-1 more than the increase observed basally due to Rho kinase inhibition. Thus, inhibition of ROCK does not appear to rescue IGF-1 induced engulfment suppression. g, LR73 cells were treated with CK-666 and then assessed for uptake of liposomes in the presence of IGF-1. Data represented as mean±s.d.

FIG. 8, comprising FIGS. 8a-8e, demonstrates that Macrophages express IGF-1R and phosphorylate IGF-1R upon IGF-1 stimulation but engulf apoptotic cells at normal capacity when exposed to IGF-1 or insulin.

a,b, J774 cells (a) or LR73 cells (b) treated with 100 ng/mL mouse IGF-1 treated were assessed for their ability to engulf apoptotic thymocytes or serum-starved for 6 hours and stimulated with 100 ng/mL mouse IGF-1 and assessed for phosphorylation of IGF-1R by Western blot. c, Flow cytometry histograms of IGF-1R expression on J774 cells (left), bone marrow derived macrophages (middle), and peritoneal macrophages (right) (n=3-4). d,e, IC-21 cells treated with indicated concentrations of mouse IGF-1 (d) or human insulin (e) were assessed for their ability to engulf apoptotic thymocytes (n=2-3). Error bars represent s.d.

FIG. 9, comprising FIGS. 9a-9d, demonstrates that Production of IGF-1 by peritoneal macrophages after apoptotic cell or IL-4 stimulation correlates with new transcription.

a, Peritoneal macrophages were either untreated, stimulated with rIL-4 or apoptotic Jurkat cells and Igf1 mRNA (top panels) and IGF-1 protein in the supernatant (bottom panels) were assessed in a time course (n.d., not detected) (n=3). Data represented as mean±s.d. b, c, Lung sections from wild type mice were stained with antibodies against alveolar macrophages (Mac-2), airway epithelial cells (CC-10), and IGF-1R. d, Alveolar macrophages isolated from LysM-Cre/Igf1^{fl/fl} and littermate controls were assessed for Igf-1 mRNA expression (n=2 per group, data represented as mean±s.e.m.).

FIG. 10, comprising FIGS. 10a-10c, demonstrates that CCSP-Cre/Igf1r^{fl/fl} mice exposed to HDM have greater airway resistance and show a trend toward greater immune cell infiltration in the lungs and more apoptotic cells.

a, Total cell counts of lung CD3+CD4⁺ T-cells (left), CD3±CD4+CD44⁺ T-cells (middle), and CD3+CD4+CD69⁺ T-cells (right panel) in the lungs of CCSP-Cre/Igf1r^{wt/wt} and Igf1r^{fl/fl} mice given the full HDM course. b, Airway hyper-responsiveness to methacholine (another measure of allergen sensitivity) in the CCSP-Cre/Igf1r^{fl/fl} mice compared to control CCSP-Cre/Igf1r^{wt/wt} mice treated with HDM (n=6-8 mice per group). c, Representative histology images of cleaved caspase (CC3) staining in lung sections of mice given the full HDM course. Black arrowheads indicate positive staining. Average CC3-positive cells per mouse are quantified on the right (n=3 per group). Data represented as mean±s.e.m.

FIG. 11, comprising FIGS. 11a to 11c, demonstrates by Schematic IGF-1R deletion during the sensitization versus challenge phases of HDM administration, and demonstrates that the response of CCSP-Cre/Igf1r^{wt/wt} and Igf1r^{fl/fl} mice in regimen #2 (the challenge phase).

a, Schematic describing the different time courses for Igf1r deletion from Club cells (induced via administration of doxycycline) and for the allergen HDM exposure. b, Total cell counts of various populations in the BAL fluid of CCSP-Cre/Igf1r^{wt/wt} and CCSP-Cre/Igf1r^{fl/fl} mice given HDM as according to regimen #2. c, Total cell counts of CD3±CD4⁺ T-cells of draining lymph nodes of CCSP-Cre/Igf1r^{wt/wt} and CCSP-Cre/Igf1r^{fl/fl} mice given HDM as according to regimen #2. Data represented as mean±s.e.m.

FIG. 12, comprising FIGS. 12a-12c, demonstrates that Alveolar macrophage-derived microvesicles suppress gene expression in lung epithelial cells exposed to house dust mite extract.

a, Microvesicles (MV) were harvested from either control or IL-4 treated MH-S alveolar macrophages and then counted using qNano (n=3). b, Supernatants from IL-4 treated MH-S macrophages were assessed for IGF-1 secretion. c, BEAS-2B cells were treated with HDM either in the presence or absence of alveolar macrophage-derived microvesicles for 3 hours and then assessed for expression of FGF2, KLF4, IFIT2, and PTX3 (n=6). Data represented as mean±s.e.m.

FIG. 13, comprising FIGS. 13a-13b, schematically illustrates a Model for alveolar macrophage regulation (via IGF-1 and microvesicles) of airway epithelial cell with respect to particle uptake and response to allergens.

Exposure of airways to allergens, such as HDM, can cause apoptotic cell death as well as IL-4 and IL-13 production, from mast cells and type 2 innate lymphoid cells (ILC2s). These cytokines, along with apoptotic cells, trigger alveolar macrophages to produce IGF-1. The released IGF-1 (a) then acts on the airway epithelium to elicit two actions: first, to decrease the uptake of apoptotic cells and second to enhance the uptake of macrophage-derived microvesicles. These microvesicles (b) dampen inflammatory cytokine production by the airway epithelial cells.

FIGS. 5-13 and their subcomponents are also referred to as Extended FIGS. 1-9, respectively. The provisional patent application from which this application claims priority was based on a draft manuscript now published as Han et al., Nature, 2016, 539:570-74 entitled “Macrophages redirect phagocytosis by nonprofessional phagocytes and influence inflammation”.

### DETAILED DESCRIPTION

### Abbreviations and Acronyms

aa—amino acid

BAL—bronchoalveolar lavage

CCSP—Club cell secretory protein

CSF-2—colony stimulating factor 2

cytoD—cytochalasin D

FGF2—fibroblast growth factor 2

GM-CSF—granulocyte/macrophage colony stimulating factor

HDM—house dust mite

IFIT2—Interferon-Induced Protein with Tetratricopeptide Repeats 2

IGF-I—insulin-like growth factor 1 (also referred to as IGF-1)

IGF-1R—insulin-like growth factor 1 receptor

IGF-II—insulin-like growth factor II (also referred to as IGF-2)

IGFBP—insulin-like growth factor binding protein

IL—interleukin

IL-4—interleukin 4

IL-6—interleukin 6

IL-8—interleukin 8

IL-11—interleukin 11

ILC2—type 2 innate lymphoid cell

KLF4—Kruppel-like factor 4

MLV—multilamellar vesicles

MV—microvesicle

Pentraxin 3—PTX3

PtdSer—phosphatidylserine

TSLP—thymic stromal lymphopoietin

### Definitions

As used herein, the terms below are defined by the following meanings:

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described herein. As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

The terms “additional therapeutically active compound” or “additional therapeutic agent”, as used in the context of the present invention, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As use herein, the terms “administration of” and or “administering” a compound should be understood to mean providing a compound of the invention or a prodrug of a compound of the invention to a subject in need of treatment.

As used herein, an “agonist” is a composition of matter which, when administered to a mammal such as a human, enhances or extends a biological activity attributable to the level or presence of a target compound or molecule of interest in the mammal.

An “antagonist” is a composition of matter which when administered to a mammal such as a human, inhibits a biological activity attributable to the level or presence of a compound or molecule of interest in the mammal.

As used herein, “alleviating a disease or disorder symptom,” means reducing the severity of the symptom or the frequency with which such a symptom is experienced by a patient, or both. A “therapeutic” treatment is a treatment administered to a subject who exhibits signs of pathology for the purpose of diminishing or eliminating those signs.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

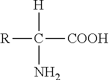

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin subunit molecules. The antibodies in the present invention may exist in a variety of forms including, for example, polyclonal antibodies, monoclonal antibodies, Fv, Fab and F(ab)2, as well as single chain antibodies and humanized antibodies.

An “antibody heavy chain,” as used herein, refers to the larger of the two types of polypeptide chains present in all antibody molecules.

An “antibody light chain,” as used herein, refers to the smaller of the two types of polypeptide chains present in all antibody molecules.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the term “secondary antibody” refers to an antibody that binds to the constant region of another antibody (the primary antibody).

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “antimicrobial agents” as used herein refers to any naturally-occurring, synthetic, or semi-synthetic compound or composition or mixture thereof, which is safe for human or animal use as practiced in the methods of this invention, and is effective in killing or substantially inhibiting the growth of microbes. “Antimicrobial” as used herein, includes antibacterial, antifungal, and antiviral agents.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands. “Binding partner,” as used herein, refers to a molecule capable of binding to another molecule.

The term “biocompatible”, as used herein, refers to a material that does not elicit a substantial detrimental response in the host.

The term “biological sample,” as used herein, refers to samples obtained from a subject, including, but not limited to, skin, hair, tissue, blood, plasma, serum, cells, sweat, saliva, feces, tissue and/or urine.

As used herein, the term “biologically active fragments” or “bioactive fragment” of the polypeptides encompasses natural or synthetic portions of the full length protein that are capable of specific binding to their natural ligand or of performing the function of the protein. For example, a “functional” or “active” biological molecule is a biological molecule in a form in which it exhibits a property by which it is characterized. A functional enzyme, for example, is one which exhibits the characteristic catalytic activity by which the enzyme is characterized.

As used herein, the term “carrier molecule” refers to any molecule that is chemically conjugated to the antigen of interest that enables an immune response resulting in antibodies specific to the native antigen.

The term “cell surface protein” means a protein found where at least part of the protein is exposed at the outer aspect of the cell membrane. Examples include growth factor receptors.

As used herein, the term “chemically conjugated,” or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Co-administer” can include simultaneous and/or sequential administration of two or more agents.

A “compound,” as used herein, refers to any type of substance or agent that is can be considered a drug or molecule, or a candidate for use as a drug or molecule, as well as combinations and mixtures of the above.

The terms “comprises”, “comprising”, and the like can have the meaning ascribed to them in U.S. Patent Law and can mean “includes”, “including” and the like. As used herein, “including” or “includes” or the like means including, without limitation.

As used herein, the term “conservative amino acid substitution” is defined herein as an amino acid exchange within one of the following five groups:
I. Small aliphatic, nonpolar or slightly polar residues:
   Ala, Ser, Thr, Pro, Gly;
II. Polar, negatively charged residues and their amides:
   Asp, Asn, Glu, Gln;
III. Polar, positively charged residues:
   His, Arg, Lys;
IV. Large, aliphatic, nonpolar residues:
   Met Leu, Ile, Val, Cys
V. Large, aromatic residues:
   Phe, Tyr, Trp

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

A “test” cell is a cell being examined.

“Cytokine,” as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets, and effector activities of these cytokines have been described.

As used herein, “decrease in one or more inflammatory response associated proteins” refers to the expression, levels, or secretion of the protein(s).

As used herein, a “derivative” of a compound refers to a chemical compound that may be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” or “therapeutically effective amount” generally means an amount which provides the desired local or systemic effect, such as enhanced performance. For example, an effective dose is an amount sufficient to affect a beneficial or desired clinical result. The dose could be administered in one or more administrations and can include any preselected amount. The precise determination of what would be considered an effective dose may be based on factors individual to each subject, including size, age, injury or disease being treated and amount of time since the injury occurred or the disease began. One skilled in the art, particularly a physician, would be able to determine what would constitute an effective dose. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “enhancer” is a DNA regulatory element that can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

A “fragment” or “segment” is a portion of an amino acid sequence, comprising at least one amino acid, or a portion of a nucleic acid sequence comprising at least one nucleotide. The terms “fragment” and “segment” are used interchangeably herein. As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, at least about 100 to about 200 nucleotides, at least about 200 nucleotides to about 300 nucleotides, at least about 300 to about 350, at least about 350 nucleotides to about 500 nucleotides, at least about 500 to about 600, at least about 600 nucleotides to about 620 nucleotides, at least about 620 to about 650, and or the nucleic acid fragment will be greater than about 650 nucleotides in length.

As used herein, a “functional” biological molecule is a biological molecule in a form in which it exhibits a property by which it is characterized. A functional enzyme, for example, is one which exhibits the characteristic catalytic activity by which the enzyme is characterized.

As used herein, “health care provider” includes either an individual or an institution that provides preventive, curative, promotional, or rehabilitative health care services to a subject, such as a patient.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul [50; 1990]), modified as in Karlin and Altschul [51; 1993]. This algorithm is incorporated into the NBLAST and XBLAST programs of Altschul, et al. [52], and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. [53]. Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “induction of apoptosis” means a process by which a cell is affected in such a way that it begins the process of programmed cell death, which is characterized by the fragmentation of the cell into membrane-bound particles that are subsequently eliminated by the process of phagocytosis.

An “inflammatory response in an epithelial cell” refers to the genes and proteins being turned on or, in the case of proteins, also to their release, when the epithelia cell is exposed to a cell or molecule that stimulates the response.

As used herein, the term “inhaler” refers both to devices for nasal and pulmonary administration of a drug, e.g., in solution, powder and the like. For example, the term “inhaler” is intended to encompass a propellant driven inhaler, such as is used to administer antihistamine for acute asthma attacks, and plastic spray bottles, such as are used to administer decongestants.

The term “inhibit,” as used herein, refers to the ability of a compound, agent, or method to reduce or impede a described function, level, activity, rate, etc., based on the context in which the term “inhibit” is used. Preferably, inhibition is by at least 10%, more preferably by at least 25%, even more preferably by at least 50%, and most preferably, the function is inhibited by at least 75%. The term “inhibit” is used interchangeably with “reduce” and “block.”

The term “inhibiting a protein” means to inhibit the activity, levels, or expression of the protein or gene and is interpreted based on the context in which it is used. In one aspect, it refers to inhibiting its signaling activity by inhibiting it from binding with a ligand. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting or applying” includes administration of a compound of the invention by any number of routes and means including, but not limited to, topical, oral, buccal, intravenous, intramuscular, intra-arterial, intramedullary, intrathecal, intraventricular, transdermal, subcutaneous, intraperitoneal, intranasal, intratracheal, enteral, topical, sublingual, vaginal, ophthalmic, pulmonary, or rectal means.

As used herein, an “instructional material” includes a publication, a recording, a diagram, or any other medium of expression which can be used to communicate the usefulness of the peptide or antibody of the invention in the kit for diagnosing or effecting alleviation of the various diseases or disorders recited herein.

Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

The term “isolated” refers to a compound, including antibodies, nucleic acids or proteins/peptides, or cell that has been separated from at least one component which naturally accompanies it.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

A “ligand” is a compound that specifically binds to a target receptor.

A “receptor” is a compound that specifically binds to a ligand.

A ligand or a receptor (e.g., an antibody) “specifically binds to” or “is specifically immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988, Antibodies, A Laboratory Manual, Cold Spring Harbor Publications, New York) for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions, e.g., a nucleic acid molecule that hybridizes to one complementary sequence at the 5′ end and to another complementary sequence at the 3′ end, thus joining two non-complementary sequences.

The term “macrophages are in close proximity” to a cell means that the macrophages are close enough that anything they release such as IGF-I or microvesicles can come in contact easily with a target cell, such as a non-professional phagocyte.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “nasal administration” in all its grammatical forms refers to administration of at least one compound of the invention through the nasal mucous membrane to the bloodstream for systemic delivery of at least one compound of the invention. The advantages of nasal administration for delivery are that it does not require injection using a syringe and needle, it avoids necrosis that can accompany intramuscular administration of drugs, and trans-mucosal administration of a drug is highly amenable to self-administration. “Nasal administration” is also referred to as “intranasal administration”.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is also contemplated to include, but is not limited to, subcutaneous, intraperitoneal, intramuscular, intrasternal injection, and kidney dialytic infusion techniques.

The term “peptide” typically refers to short polypeptides.

The term “per application” as used herein refers to administration of a drug or compound to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

“Pharmaceutically acceptable” means physiologically tolerable, for either human or veterinary application.

As used herein, “pharmaceutical compositions” include formulations for human and veterinary use.

“Plurality” means at least two.

The term “prevent,” as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “prophylactic” treatment is a treatment administered to a subject who does not exhibit signs of a disease or exhibits only early signs of the disease for the purpose of decreasing the risk of developing pathology associated with the disease.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. In particular, purified sperm cell DNA refers to DNA that does not produce significant detectable levels of non-sperm cell DNA upon PCR amplification of the purified sperm cell DNA and subsequent analysis of that amplified DNA. A “significant detectable level” is an amount of contaminate that would be visible in the presented data and would need to be addressed/explained during analysis of the forensic evidence.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

The term “regulate” refers to either stimulating or inhibiting a function or activity of interest.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-β-galactoside to the medium (Gerhardt et al., eds., 1994, Methods for General and Molecular Bacteriology, American Society for Microbiology, Washington, D.C., p. 574).

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By the term “specifically binds to”, as used herein, is meant when a compound or ligand functions in a binding reaction or assay conditions which is determinative of the presence of the compound in a sample of heterogeneous compounds.

The term “standard,” as used herein, refers to something used for comparison. For example, it can be a known standard agent or compound which is administered and used for comparing results when administering a test compound or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker. Standard can also refer to a healthy individual.

A “subject” is a vertebrate, including a mammal, such as a human. Mammals include, but are not limited to, humans, farm animals, sport animals, and pets.

A “subject in need thereof” is a patient, animal, mammal, or human, who will benefit from the method of this invention—for example, one who is at risk of inflammation or who has inflammation, has been infected with a pathogen, exposed to an allergen, been injured, etc. Furthermore, based on the teachings of the present invention a clinician or other professional can determine if a preventive treatment may be necessary.

The term “substantially pure” describes a compound, e.g., a protein or polypeptide, cell or nucleic acid that has been separated from components which naturally accompany it. Typically, a compound is substantially pure when at least 10%, including at least 20%, at least 50%, at least 60%, at least 75%, at least 90%, at least 95%, at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

As used herein, a “substantially homologous amino acid sequences” or “substantially identical amino acid sequences” includes those amino acid sequences which have at least about 92%, or at least about 95% homology or identity, including at least about 96% homology or identity, including at least about 97% homology or identity, including at least about 98% homology or identity, and at least about 99% or more homology or identity to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially homologous nucleic acid sequence” or “substantially identical nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In one embodiment, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 92%, 95%, 99% or more. Substantial identity of nucleic acid sequences can be determined by comparing the sequence identity of two sequences, for example by physical/chemical methods (i.e., hybridization) or by sequence alignment via computer algorithm.

Suitable nucleic acid hybridization conditions to determine if a nucleotide sequence is substantially similar to a reference nucleotide sequence are: 7% sodium dodecyl sulfate SDS, 0.5 M NaPO4, 1 mM EDTA at 50° C. with washing in 2× standard saline citrate (SSC), 0.1% SDS at 50° C.; preferably in 7% (SDS), 0.5 M NaPO4, 1 mM EDTA at 50° C. with washing in 1×SSC, 0.1% SDS at 50° C.; preferably 7% SDS, 0.5 M NaPO4, 1 mM EDTA at 50° C. with washing in 0.5×SSC, 0.1% SDS at 50° C.; and more preferably in 7% SDS, 0.5 M NaPO4, 1 mM EDTA at 50° C. with washing in 0.1×SSC, 0.1% SDS at 65° C. Suitable computer algorithms to determine substantial similarity between two nucleic acid sequences include, GCS program package. The default settings provided with these programs are suitable for determining substantial similarity of nucleic acid sequences for purposes of the present invention.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “transgenic mammal” means a mammal, the germ cells of which comprise an exogenous nucleic acid.

As used herein, a “transgenic cell” is any cell that comprises a nucleic acid sequence that has been introduced into the cell in a manner that allows expression of a gene encoded by the introduced nucleic acid sequence.

As used herein, “treat,” “treating”, or “treatment” includes treating, ameliorating, or inhibiting an injury or disease related condition or a symptom of an injury or disease related condition. In one embodiment the disease, injury or disease related condition or a symptom of an injury or disease related condition is prevented; while another embodiment provides prophylactic treatment of the injury or disease related condition or a symptom of an injury or disease related condition.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like. “Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### Embodiments

One or more proteins of the invention, or biologically active fragments or homologs thereof, can be administered to a subject in need thereof. Additionally, instead of administering the protein(s), an expression vector comprising a nucleic acid sequence encoding the protein, or a biologically active fragment thereof, can be administered.

The proteins and peptides of the present invention may be purchased in some cases and can be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions that will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method that utilized tert-butyloxcarbonyl as the α-amino protecting group, and the FMOC method which utilizes 9-fluorenylmethyloxcarbonyl to protect the α-amino of the amino acid residues, both methods of which are well-known by those of skill in the art.

To ensure that the proteins or peptides obtained from either chemical or biological synthetic techniques is the desired peptide, analysis of the peptide composition should be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide can be purified to remove contaminants. In this regard, it will be appreciated that the peptide will be purified to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Substantially pure peptide obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

This invention encompasses methods of screening compounds to identify those compounds that act as agonists (stimulate) or antagonists (inhibit) of the protein interactions and pathways described herein. Screening assays for antagonist compound candidates are designed to identify compounds that bind or complex with the peptides described herein, or otherwise interfere with the interaction of the peptides with other cellular proteins. Such screening assays will include assays amenable to high-throughput screening of chemical libraries, making them particularly suitable for identifying small molecule drug candidates.

The assays can be performed in a variety of formats, including protein-protein binding assays, biochemical screening assays, high-throughput assays, immunoassays, and cell-based assays, which are well characterized in the art.

All assays for antagonists are common in that they call for contacting the compound or drug candidate with a peptide identified herein under conditions and for a time sufficient to allow these two components to interact.

In binding assays, the interaction is binding and the complex formed can be isolated or detected in the reaction mixture. In a particular embodiment, one of the peptides of the complexes described herein, or the test compound or drug candidate is immobilized on a solid phase, e.g., on a microtiter plate, by covalent or non-covalent attachments. Non-covalent attachment generally is accomplished by coating the solid surface with a solution of the peptide and drying. Alternatively, an immobilized antibody, e.g., a monoclonal antibody, specific for the peptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with, but does not bind to a particular peptide identified herein, its interaction with that peptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Complete kits for identifying protein-protein interactions between two specific proteins using the two-hybrid technique are available. This system can also be extended to map protein domains involved in specific protein interactions as well as to pinpoint amino acid residues that are crucial for these interactions.

Compounds that interfere with the interaction of a peptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

Other assays and libraries are encompassed within the invention, such as the use of Phylomers® and reverse yeast two-hybrid assays (see Watt, 2006, Nature Biotechnology, 24:177; Watt, U.S. Pat. No. 6,994,982; Watt, U.S. Pat. Pub. No. 2005/0287580; Watt, U.S. Pat. No. 6,510,495; Barr et al., 2004, J. Biol. Chem., 279:41:43178-43189; the contents of each of these publications is hereby incorporated by reference herein in their entirety). Phylomers® are derived from sub domains of natural proteins, which makes them potentially more stable than conventional short random peptides. Phylomers® are sourced from biological genomes that are not human in origin. This feature significantly enhances the potency associated with Phylomers® against human protein targets. Phylogica's current Phylomer® library has a complexity of 50 million clones, which is comparable with the numerical complexity of random peptide or antibody Fab fragment libraries. An Interacting Peptide Library, consisting of 63 million peptides fused to the B42 activation domain, can be used to isolate peptides capable of binding to a target protein in a forward yeast two hybrid screen. The second is a Blocking Peptide Library made up of over 2 million peptides that can be used to screen for peptides capable of disrupting a specific protein interaction using the reverse two-hybrid system.

The Phylomer® library consists of protein fragments, which have been sourced from a diverse range of bacterial genomes. The libraries are highly enriched for stable subdomains (15-50 amino acids long). This technology can be integrated with high throughput screening techniques such as phage display and reverse yeast two-hybrid traps.

The present application discloses compositions and methods for regulating the proteins described herein, and those not disclosed which are known in the art are encompassed within the invention. For example, various modulators/effectors are known, e.g. antibodies, biologically active nucleic acids, such as antisense molecules, RNAi molecules, or ribozymes, aptamers, peptides or low-molecular weight organic compounds recognizing said polynucleotides or polypeptides. The present invention also provides nucleic acids encoding peptides, proteins, and antibodies of the invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

It is not intended that the present invention be limited by the nature of the nucleic acid employed. The target nucleic acid may be native or synthesized nucleic acid. The nucleic acid may be from a viral, bacterial, animal or plant source. The nucleic acid may be DNA or RNA and may exist in a double-stranded, single-stranded or partially double-stranded form. Furthermore, the nucleic acid may be found as part of a virus or other macromolecule. See, e.g., Fasbender et al., 1996, J. Biol. Chem. 272:6479-89 (polylysine condensation of DNA in the form of adenovirus).

In some circumstances, as where increased nuclease stability is desired, nucleic acids having modified internucleoside linkages may be preferred. Nucleic acids containing modified internucleoside linkages may also be synthesized using reagents and methods that are well known in the art. For example, methods for synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH2-S—CH2), diinethylene-sulfoxide (—CH2-SO—CH2), dimethylene-sulfone (—CH2-SO2-CH2), 2′-O-alkyl, and 2′-deoxy2′-fluoro phosphorothioate internucleoside linkages are well known in the art (see Uhlmann et al., 1990, Chem. Rev. 90:543-584; Schneider et al., 1990, Tetrahedron Lett. 31:335 and references cited therein).

The nucleic acids may be purified by any suitable means, as are well known in the art. For example, the nucleic: acids can be purified by reverse phase or ion exchange HPLC, size exclusion chromatography or gel electrophoresis. Of course, the skilled artisan will recognize that the method of purification will depend in part on the size of the DNA to be purified. The term nucleic acid also specifically includes nucleic acids composed of bases other than the five biologically occurring bases (adenine, guanine, thymine, cytosine, and uracil).

The present invention is also directed to pharmaceutical compositions comprising the compounds of the present invention. More particularly, such compounds can be formulated as pharmaceutical compositions using standard pharmaceutically acceptable carriers, fillers, solublizing agents and stabilizers known to those skilled in the art.

When used in vivo for therapy, the proteins of the invention, as well as biologically active fragments and homologs thereof, are administered to the subject in therapeutically effective amounts (i.e., amounts that have a desired therapeutic effect). In one aspect, they will be administered parenterally.

In accordance with one embodiment, a method of treating a subject in need of treatment is provided. The method comprises administering a pharmaceutical composition comprising at least one compound of the present invention to a subject in need thereof. Compounds identified by the methods of the invention can be administered with known compounds or other medications as well.

The pharmaceutical compositions useful for practicing the invention may be administered to deliver a dose of between 1 ng/kg/day and 100 mg/kg/day.

The invention encompasses the preparation and use of pharmaceutical compositions comprising a compound useful for treatment of the diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically, dosages of the compound of the invention which may be administered to an animal, preferably a human, range in amount from 1 μg to about 100 g per kilogram of body weight of the subject. While the precise dosage administered will vary depending upon any number of factors, including, but not limited to, the type of animal and type of disease state being treated, the age of the subject and the route of administration. In one aspect, the dosage of the compound will vary from about 1 mg to about 10 g per kilogram of body weight of the subject. In another aspect, the dosage will vary from about 10 mg to about 1 g per kilogram of body weight of the subject.

The compound may be administered to a subject as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the condition or disease being treated, the type and age of the subject, etc.

The invention is also directed to methods of administering the compounds of the invention to a subject. In one embodiment, the invention provides a method of treating a subject by administering compounds identified using the methods of the invention. Pharmaceutical compositions comprising the present compounds are administered to an individual in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means.

For oral administration, the active ingredient can be administered in solid dosage forms, such as capsules, tablets, and powders, or in liquid dosage forms, such as elixirs, syrups, and suspensions. Active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate, and the like. Examples of additional inactive ingredients that may be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The invention also includes a kit comprising the composition of the invention and an instructional material which describes adventitially administering the composition to a cell or a tissue of a mammal. In another embodiment, this kit comprises a (preferably sterile) solvent suitable for dissolving or suspending the composition of the invention prior to administering the compound to the mammal.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviation the diseases or disorders in a cell or a tissue of a mammal. The instructional material of the kit of the invention may, for example, be affixed to a container which contains the peptide of the invention or be shipped together with a container which contains the peptide. Alternatively, the instructional material may be shipped separately from the container with the intention that the instructional material and the compound be used cooperatively by the recipient.

Other techniques known in the art may be used in the practice of the present invention, including those described in international patent application WO 2006/091535 (PCT/US2006/005970), the entirety of which is incorporated by reference herein.

It will be appreciated, of course, that the proteins or peptides of the invention may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring or non-standard synthetic amino acids. The peptides of the invention are not limited to products of any of the specific exemplary processes listed herein.

The invention includes the use of beta-alanine (also referred to as β-alanine, β-Ala, bA, and βA, having the structure:

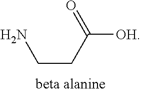

Sequences are provided herein which use the symbol “PA”, but in the Sequence Listing submitted herewith “PA” is provided as “Xaa” and reference in the text of the Sequence Listing indicates that Xaa is beta alanine.

Peptides useful in the present invention, such as standards, or modifications for analysis, may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well-known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide may be purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high performance liquid chromatography (HPLC) using an alkylated silica column such as C4-, C8- or C18-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

As discussed, modifications or optimizations of peptide ligands of the invention are within the scope of the application. Modified or optimized peptides are included within the definition of peptide binding ligand. Specifically, a peptide sequence identified can be modified to optimize its potency, pharmacokinetic behavior, stability and/or other biological, physical and chemical properties.

Amino Acid Substitutions

In certain embodiments, the disclosed methods and compositions may involve preparing peptides with one or more substituted amino acid residues. In various embodiments, the structural, physical and/or therapeutic characteristics of peptide sequences may be optimized by replacing one or more amino acid residues.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art.

For example, one can make the following isosteric and/or conservative amino acid changes in the parent polypeptide sequence with the expectation that the resulting polypeptides would have a similar or improved profile of the properties described above:

Substitution of alkyl-substituted hydrophobic amino acids: including alanine, leucine, isoleucine, valine, norleucine, S-2-aminobutyric acid, S-cyclohexylalanine or other simple alpha-amino acids substituted by an aliphatic side chain from C1-10 carbons including branched, cyclic and straight chain alkyl, alkenyl or alkynyl substitutions.

Substitution of aromatic-substituted hydrophobic amino acids: including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-, 3- or 4-aminophenylalanine, 2-, 3- or 4-chlorophenylalanine, 2-, 3- or 4-methylphenylalanine, 2-, 3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2,3, or 4-biphenylalanine, 2′,-3′,- or 4′-methyl-2, 3 or 4-biphenylalanine, and 2- or 3-pyridylalanine.

Substitution of amino acids containing basic functions: including arginine, lysine, histidine, ornithine, 2,3-diaminopropionic acid, homoarginine, alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of acidic amino acids: including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids.

Substitution of side chain amide residues: including asparagine, glutamine, and alkyl or aromatic substituted derivatives of asparagine or glutamine.

Substitution of hydroxyl containing amino acids: including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids may be considered (Kyte & Doolittle, 1982, J. Mol. Biol., 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids can include various hydropathic indices. In one aspect, the hydropathic indices are within +/−2, in another they are within +/−1, and in one aspect, they are within +/−0.5.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4) In one aspect, the replacement of amino acids with others of similar hydrophilicity is provided by the invention.

Other considerations include the size of the amino acid side chain. For example, it would generally not be preferable to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see, e.g., Chou & Fasman, 1974, Biochemistry, 13:222-245; 1978, Ann. Rev. Biochem., 47: 251-276; 1979, Biophys. J., 26:367-384).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) leu, ile, val; Arg (R) gln, asn, lys; Asn (N) his, asp, lys, arg, gln; Asp (D) asn, glu; Cys (C) ala, ser; Gln (Q) glu, asn; Glu (E) gln, asp; Gly (G) ala; His (H) asn, gln, lys, arg; Ile (I) val, met, ala, phe, leu; Leu (L) val, met, ala, phe, ile; Lys (K) gln, asn, arg; Met (M) phe, ile, leu; Phe (F) leu, val, ile, ala, tyr; Pro (P) ala; Ser (S), thr; Thr (T) ser; Trp (W) phe, tyr; Tyr (Y) trp, phe, thr, ser; Val (V) ile, leu, met, phe, ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. (See, e.g., PROWL Rockefeller University website). For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. (Id.) Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix (Idem.)

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

In other embodiments, therapeutic agents, including, but not limited to, cytotoxic agents, anti-angiogenic agents, pro-apoptotic agents, antibiotics, hormones, hormone antagonists, chemokines, drugs, prodrugs, toxins, enzymes or other agents may be used as adjunct therapies when using the antibody/peptide ligand complexes described herein.

Nucleic acids useful in the present invention include, by way of example and not limitation, oligonucleotides and polynucleotides such as antisense DNAs and/or RNAs; ribozymes; DNA for gene therapy; viral fragments including viral DNA and/or RNA; DNA and/or RNA chimeras; mRNA; plasmids; cosmids; genomic DNA; cDNA; gene fragments; various structural forms of DNA including single-stranded DNA, double-stranded DNA, supercoiled DNA and/or triple-helical DNA; Z-DNA; and the like. The nucleic acids may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, OLIGONUCLEOTIDE SYNTHESIS: A PRACTICAL APPROACH (IRL Press, Oxford, England)). RNAs may be produce in high yield via in vitro transcription using plasmids such as SP65 (Promega Corporation, Madison, Wis.).

The invention further provides cells transfected with the nucleic acid containing an enhancer/promoter combination of the invention.

Promoters may be coupled with other regulatory sequences/elements which, when bound to appropriate intracellular regulatory factors, enhance (“enhancers”) or repress (“repressors”) promoter-dependent transcription. A promoter, enhancer, or repressor, is said to be “operably linked” to a transgene when such element(s) control(s) or affect(s) transgene transcription rate or efficiency. For example, a promoter sequence located proximally to the 5′ end of a transgene coding sequence is usually operably linked with the transgene. As used herein, term “regulatory elements” is used interchangeably with “regulatory sequences” and refers to promoters, enhancers, and other expression control elements, or any combination of such elements.

Promoters are positioned 5′ (upstream) to the genes that they control. Many eukaryotic promoters contain two types of recognition sequences: TATA box and the upstream promoter elements. The TATA box, located 25-30 bp upstream of the transcription initiation site, is thought to be involved in directing RNA polymerase II to begin RNA synthesis as the correct site. In contrast, the upstream promoter elements determine the rate at which transcription is initiated. These elements can act regardless of their orientation, but they must be located within 100 to 200 bp upstream of the TATA box.

Enhancer elements can stimulate transcription up to 1000-fold from linked homologous or heterologous promoters. Enhancer elements often remain active even if their orientation is reversed (Li et al., J. Bio. Chem. 1990, 266: 6562-6570). Furthermore, unlike promoter elements, enhancers can be active when placed downstream from the transcription initiation site, e.g., within an intron, or even at a considerable distance from the promoter (Yutzey et al., Mol. and Cell. Bio. 1989, 9:1397-1405).

It is known in the art that some variation in this distance can be accommodated without loss of promoter function. Similarly, the positioning of regulatory elements with respect to the transgene may vary significantly without loss of function. Multiple copies of regulatory elements can act in concert. Typically, an expression vector comprises one or more enhancer sequences followed by, in the 5′ to 3′ direction, a promoter sequence, all operably linked to a transgene followed by a polyadenylation sequence.

The present invention further relies on the fact that many enhancers of cellular genes work exclusively in a particular tissue or cell type. In addition, some enhancers become active only under specific conditions that are generated by the presence of an inducer such as a hormone or metal ion. Because of these differences in the specificities of cellular enhancers, the choice of promoter and enhancer elements to be incorporated into a eukaryotic expression vector is determined by the cell type(s) in which the recombinant gene is to be expressed.

In one aspect, the regulatory elements of the invention may be heterologous with regard to each other or to a transgene, that is, they may be from different species. Furthermore, they may be from species other than the host, or they also may be derived from the same species but from different genes, or they may be derived from a single gene.

Additional types of compounds can be administered to treat further the addiction-related diseases and disorders or to treat other diseases and disorders. The additional types of compounds include, but are not limited to, adrenocortical steroids, amino acids, analeptics, analgesics, anesthetics, antihypertensives, antibiotics, anti-inflammatories, antimicrobials, antinauseants, blood glucose regulators, cardiovascular agents, hormones, relaxants, sedative-hypnotics, stimulants, thyroid hormones, thyroid inhibitors, thyromimetics, cerebral ischemia agents, vasoconstrictors, and vasodilators.

The present invention provides for multiple methods for delivering the compounds of the invention. The compounds may be provided, for example, as pharmaceutical compositions in multiple formats as well, including, but not limited to, tablets, capsules, pills, lozenges, syrups, ointments, creams, elixirs, suppositories, suspensions, inhalants, injections (including depot preparations), and liquids. The present invention further encompasses the use of combination viral or gene therapy, and pharmacotherapy.

Suitable preparations include injectables, either as liquid solutions or suspensions, however, solid forms suitable for solution in, suspension in, liquid prior to injection, may also be prepared. The preparation may also be emulsified, or the polypeptides encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants.

The present invention further encompasses kits.

Compositions of the present invention may be presented in a pack or dispenser device, such as an FDA approved kit, which may contain one or more unit dosage forms containing the therapeutic compound as described herein.

In some embodiments, the kit may include a therapeutic compound (as described herein), metal or plastic foil, such as a blister pack, a dispenser device or an applicator, tubes, buffers, and instructions for administration. The various reagent components of the kits may be present in separate containers, or some or all of them may be pre-combined into a reagent mixture in a single container, as desired. The dispenser device or applicator may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S.

Food and Drug Administration for prescription drugs or of an approved product insert.

### Examples

Methods

### Mice

C57BL/6J, Igf1r^{fl/fl}, and Igf1^{fl/fl} mice were obtained from Jackson Laboratories. CCSP-_{ntTA/tet( )}-Cre were kindly provided by Dr. Jeffrey Whitsett at Cincinnati Children's Hospital. To generate IGF-1R deletion in Club cells, we crossed CCSP-_{ntTA/tet( )}-Cre mice to Igf1^{fl/fl}. To achieve deletion, mice were given doxycycline (1 mg/mL) in drinking water containing 0.4% sucrose for at least 7 days prior to beginning of allergen administration, unless otherwise noted. We also crossed Igf1^{fl/fl} mice to LysM-Cre mice to conditionally delete Igf1 in the myeloid lineage. We have previously reported on the generation of CCSP-Cre/YFP mice.

For all in vivo experiments, except generation of apoptotic thymocytes, mice between the ages of 8 and 12 weeks were used. No blinding was performed for in vivo experiments. Mice were allocated to experimental groups based on genotype and age-matching. Female and male mice were used for all experiments, except for in vivo engulfment assays in which only male mice were used. Sample size was estimated based on a previous publication on airway inflammation and apoptotic cell clearance³. All animal procedures were performed according to the protocols provided by the Institutional Animal Care and Use Committee (IACUC) of the University of Virginia.

### Induction of Airway Inflammation

Mice were given drinking water containing doxycycline (1 mg/mL) seven days prior to first HDM administration. Mice were primed intranasally with 10 μg of low endotoxin house dust mite extraction (Indoor Biotechnologies) on days 0, 2, 4 and then challenged intranasally on days 10, 12, and 14. On day 16, mice were harvested and analyzed for eosinophilic airway inflammation. Alternatively, mice were given three doses of low endotoxin HDM on days 0, 2, and 4 and analyzed on day 6 (“sensitization phase”). For the “challenge phase,” mice that had not received any doxycycline were given three doses of low endotoxin HDM on days 0, 2 and 4, then given doxycycline (via drinking water) from day 4 until the mice were analyzed. These mice were also challenged intranasally with three doses of low endotoxin HDM on days 10, 12, and 14 and analyzed on day 16.

### Collection of BAL Fluid, Lymph Nodes, and Lung

For airway inflammation experiments, 0.8 mL of PBS were delivered intratracheally through a cannula. Recovered BAL fluid was centrifuged and the supernatant was frozen at −80° C. for subsequent Luminex analysis. Collected cells were stained for surface markers to distinguish macrophages, neutrophils, T-cells, and eosinophils. For lung harvests, mice were perfused through the right ventricle with PBS and the lungs were carefully excised and placed in type 2 collagenase (Worthington Biochemical Corporation) dissolved in HBSS containing Ca²⁺ and Mg²⁺. Lungs were minced and then incubated at 37° C. for one hour, with vigorous pipetting every 15 minutes to separate the tissue. The lung homogenate was then passed through a 70 μm nylon strainer, spun down and treated with red blood cell lysis buffer (Sigma-Aldrich) for 5 minutes. The cells were then washed and resuspended in PBS containing 0.1% BSA. Draining lymph nodes were carefully extracted, and a single cell suspension was made by passage through a 70 μm nylon strainer using the flat end of a syringe. Cells were washed and then resuspended in PBS containing 0.1% BSA.

### Cell Staining and Total Cell Numbers

The collected cells were stained for macrophages, neutrophils, T-cells, and eosinophils using the following markers: CD11c (eBioscience, cl. N418), Siglec F (BD Biosciences, cl. E50-2440), Ly6G (eBioscience, clone 1A8), CD11b (eBioscience, cl. M1/70), F4/80 (eBioscience, cl. BM8), CD3 (eBioscience, cl. 145-2C11), CD4 (eBioscience, cl. RM4-5), CD44 (eBioscience, cl. IM7), CD69 (eBioscience, cl. H1.2F3). Absolute cell numbers were determined using AccuCount Particles (Spherotech). Flow cytometry data was collected on FACS Canto I (Becton Dickinson) and analyzed with FlowJo (Treestar, Inc).

### Microscopy and Histology

For hematoxylin and eosin and Periodic acid Schiff staining of lung sections, mice were perfused with PBS and a cannula inserted into the trachea. The lungs were gently inflated with 10% formalin at a constant fluid pressure at 25 cm. The trachea was tied off and the entire heart and lung were removed and placed in 10% formalin. Lungs were paraffin embedded, sectioned and stained by HistoTox Labs (Boulder, Colo.). Additional lung sections were embedded and sectioned by Research Histology Core at University of Virginia and the immunohistochemical staining for IGF-1R, and cleaved caspase 3 was performed by the University of Virginia Biorepository and Tissue Research Facility. Approximately 6-10 images were taken per mice, with a total of 3-4 mice per group, and blindly scored by two independent scorers for inflammation, PAS staining, and cleaved caspase 3 positive cells. Immunofluorescence staining of lung sections was performed at the University of Virginia Cardiovascular Research Center Histological Services.

### Airway Hypersensitivity

Mice were anesthetized and given a tracheotomy tube that delivered increasing concentrations of aerosolized methacholine. The tracheotomy tube in turn was connected to the inspiratory and expiratory ports of a volume-cycled ventilator (flexiVent; SCIREQ Scientific). Airway resistance was measured at baseline and after each dose of methacholine.

### Macrophage Isolation

To obtain bone-marrow derived macrophages, femurs were removed from 8 week old mice and flushed with 5 mL of sterile PBS containing 5% FBS. The cell suspension was centrifuged, treated with red blood cell lysis buffer, washed, and then plated onto sterile petri dishes in DMEM containing 10% L929 media, 10% FBS and 1% penicillin/streptomycin/glutamine (PSQ). Media was replenished every 2 to 3 days and differentiated cells were used at day 6 post-harvest. Resident peritoneal macrophages were obtained by flushing the peritoneal cavity of mice with 10 mL of cold PBS containing 5% FBS. Collected cells were spun down, resuspended in X-VIVO 10 (Lonza) and plated at a concentration of 3×10⁵ cells per well in a 24 well plate for IGF-1 secretion assays, and 5×10⁵ per well in a 24 well plate for engulfment assays. Floating cells were washed the next day and remaining peritoneal macrophages were used 2 days after isolation. Alveolar macrophages were isolated by flushing the lungs with 1 mL of cold PBS instilled intratracheally (five flushes). Collected cells were centrifuged, resuspended in F12K media containing 10% FBS and 1% PSQ, and seeded at 1×10⁵ cells per well in a 48-well plate. All floating cells were washed away the next day and remaining cells were used in assays 2 days after isolation.

### Microvesicle Isolation

MH-S, mouse alveolar macrophages, or primary mouse alveolar macrophages were seeded. After adherence, the media was replaced with 0.22 μm filtered media to remove any contaminating microvesicles. After an overnight incubation, the supernatant was harvested and spun at 5000×g to remove cell debris and apoptotic bodies. The pellet was discarded and the resulting supernatant was filtered through 0.8 μm filter and spun again at 17,000×g. The pelleted microvesicles were then washed with HBSS and then spun again at 17,000×g. For engulfment assays, microvesicles were stained with TAMRA for 20 minutes and then added to BEAS-2B cells for 90 minutes. For flow cytometry, purified particles were stained with CD11c, Siglec F, and Annexin V (BD Biosciences, Cat. No. 550475) and processed on Imagestream™ imaging flow cytometer (Amnis). Microvesicle size distribution was characterized using qNano (IZON Science) with a NP400 membrane and at least 500 particles were counted. Microvesicles were prepared for cryo-electron microscopy using standard methods and imaged on an FEI TF20.

### In Vivo Cytokine or Apoptotic Cell Administration

Mice were administered intranasally with 1 μg of recombinant mouse IL-4, IL-5, or IL-13 (eBiosciences), 1×10⁶ apoptotic Jurkat cells, or PBS as control, for two consecutive days. On the third day, BAL fluid was recovered and centrifuged; the supernatant was stored at −80° C. for subsequent cytokine analysis.

### Cytokine and IGF-1 Analysis

IL-4, IL-5, IL-6, and CCL-11/Eotaxin-1 in the BAL fluid of mice that were sensitized with HDM (regimen #1) were quantified by a multiplex Luminex performed by the University of Virginia Flow Cytometry Core Facility. Secretion of IGF-1 from J774 cells, peritoneal macrophages, and BAL fluids, as well as TSLP from BAL fluids, were measured by ELISA (R&D Systems).

### In Vitro Cell Systems

LR73 (hamster fibroblasts), SVEC-40 (mouse endothelial cells), BEAS-2B (human bronchial epithelial cells, ATCC #CRL-9609), 16HBE14o-cells (human bronchial epithelial cells), MH-S (mouse alveolar macrophage cells ATCC #CRL-2019), Jurkat (human T-cells) were either available in the laboratory or obtained from ATCC, with the latter tested for mycoplasma.

### In Vitro Engulfment Assay

LR73, SVEC-40, BEAS-2B, and 16HBE14o-cells were seeded in a 24 well plate. Thymocytes were isolated from 4-6 week old mice and induced to undergo apoptosis with dexamethasone. Thymocytes or prepared microvesicles were then stained with either CypHer5E (GE Healthcare, PA15401) or TAMRA (Invitrogen, C-1171). LR73 and SVEC-40 cells were incubated with apoptotic thymocytes at a 1:10 phagocyte to target ratio, BEAS-2B at a 1:5 phagocyte to target ratio, and 16HBE14o—at a 1:20 phagocyte to target ratio for 2 hours. Mouse IGF-I (Sigma-Aldrich), human IGF-I (Sigma-Aldrich), human IGF-II (Sigma-Aldrich) were added to the phagocytes at the same time as addition of apoptotic targets. For IGFBP3 studies, IGFBP3 (Sigma-Aldrich) was added to media or supernatant from J774 cells for one hour to allow IGFBP3 to bind to any available IGF-I, then the mix is added to phagocytes along with apoptotic targets. For all pharmacological studies, phagocytes were pre-incubated with the compounds listed below for one hour prior to addition of apoptotic targets: cytochalasin D (Sigma-Aldrich, C8273, 1 μM), Latrunculin A (Tocris, 3973, 150 nM), CK-666 (Tocris, 3950, 25 μM-100 μM), OSI-906 (Selleckchem, S1091, 5 nM-40 nM), NVP-AEW541 (Selleckchem, S1034, 25 nM-100 nM), Rapamycin (Sigma-Aldrich, R0395, 10 μM-1 mM), MK-2206 2HCl (Selleckchem, S1078, 10 nM-1 U0126-EtOH (Selleckchem, S1102, 8 μM-5 nM), Wortmannin (Sigma-Aldrich, W1628, 50 nM-200 nM), Y27632 (Calbiochem, 688000, 3.75 μM-15 μM), or GSK269962 (Tocris, 4009, 80 nM-2 μM). Phagocytes were examined to ensure no gross morphological changes occurred due to drug treatment. Targets were then washed off three times with PBS, and the cells were dissociated from the plate with trypsin and the engulfment assessed by flow cytometry.

### In Vivo Engulfment Assay

CCSP-Cre/YFP mice were administered PBS or 1 μg of IGF-1 intranasally. One hour later, 100 million CypHer5E-labelled apoptotic thymocytes with or without IGF-I were injected intranasally for 3.5 hours. The BAL fluid was harvested and the lungs excised, minced, and digested into a single cell suspension. The cells were then stained with appropriate markers: CD11c and Siglec F for alveolar macrophage markers and EpCam (along with YFP expression) for airway epithelial cells and analyzed by flow cytometry to assess apoptotic cell uptake by the airway epithelial cells and the alveolar macrophages.

### Liposome Construction

Liposomes were prepared by dissolving the lipids (phosphatidylserine, dioleoyl phosphatidylcholine, cholesterol and the lipid DiD dye) in chloroform, evaporating chloroform under flow of argon gas in a glass vial, then subjecting the lipid layer to overnight lyophilization to remove traces of organic solvent. Then normal saline was added for hydration, and intense vortexing was preformed to prepare multilamellar vesicles (MIN). Liposomes were repeatedly filtered through a 0.2 um Nuclepore polycarbonate filter to prepare smaller particles. Particle size was verified by dynamic light scattering using Nicomp 370.

### Immunoblotting

LR73, J774, or BEAS-2B cells were seeded in a 60 mm dish at a concentration of 5×10⁵. Cells were serum-starved for 6 hours and then stimulated with 100 ng/mL of IGF-1 for various time points. Cells were lysed in RIPA buffer and used in Western blots. The blots were probed for phospho-Erk1/2 (Cell Signaling Technology, #4370), phospho-Akt (Cell Signaling Technology, #4060), phospho-IGF-1R (Cell Signaling, #3024), total Erk2 (Santa Cruz Biotechnology, #sc-154-G), total Akt (Cell Signaling Technology, #4691), total IGF-1R (Cell Signaling, #9750), and anti-B-actin-HRP (Sigma-Aldrich, #A3854) followed by chemiluminescence detection.

### Quantitative RT-PCR

Total RNA was extracted from cells using Quick-RNA Miniprep Kit (Zymo Research) or RNeasy Mini Kit (Qiagen) and cDNA was synthesized using QuantiTect Reverse Transcription Kit (Qiagen) according to manufacturers' instructions. Quantitative gene expression for mouse Igf1, human TSLP, CSF2, IL6, IL8 or housekeeping human or mouse Hprt was performed using Taqman probes (Applied Biosystems) using StepOnePlus Real Time PCR System (ABI).

### RNA-Seq

BEAS-2B cells were treated with HDM and alveolar macrophage-derived microvesicles for 3 hours. Total RNA was extracted and an mRNA library was prepared using Illumina TruSeq platform and followed by transcriptome sequencing using an Illumina NextSeq 500 cartridge. Four independent experiments were sequenced. Rv3.2.2 was used for graphical and statistical analysis and the R package DESeq2 was used for differential gene expression analysis of RNA-seq data. RNA-seq analysis was performed by the UVa Bioinformatics Core.

### Code Availability

R code used for bioinformatics analysis and heat map generation is available upon request.

### Statistical Analysis

Statistical significance was determined using GraphPad Prism 5 or 6 using unpaired Student's two-tailed t-test, one-sample t-test, one-way ANOVA or two-way ANOVA, as according to test requirements. Variance was similar between groups. No inclusion/exclusion criteria were pre-established. Grubbs' Outlier Test was used to determine outliers, which were excluded from final analysis. A p-value of <0.05 (indicated by one asterisk), <0.01 (indicated by two asterisks), or <0.001 (indicated by three asterisks) were considered significant.

### Useful Proteins

Some of the proteins used in the present application are described above. Their sequences are known and some of their precursor and fragment sequences are also known and encompassed by the present disclosure where the precursors, fragments, or homologs thereof have the activity disclosed herein. Fragments and homologs of these proteins not previously known are also encompassed by the invention. Some of these proteins and their GenBank Accession numbers are provided below.

IGF-1—Human

IGF-1 precursor, partial, 119 aa protein, Accession: CAA01955.1
IGF-1, partial, 71 aa protein, Accession: CAA01954.1
IGF-1b, 195 aa protein, Accession: CAA40093.1
IGF-1a, 153 aa protein, Accession: CAA40092.1
IGF-1A precursor, 153 aa protein, Accession: CAA24998.1
IGF-1 isoform X5, predicted, 170 aa protein, Accession: XP_016874752.1
IGF-1 isoform X4, predicted 175 aa protein, Accession: XP_016874751.1
IGF-1 isoform X3, predicted, 179 aa protein, Accession: XP_016874750.1
IGF-1 isoform X1, predicted, 212 aa protein, Accession: XP_016874748.1
IGF-1 isoform 3 preproprotein, 195 aa protein, Accession: NP_001104755.1
IGF-1 isoform 2 precursor, 137 aa protein, Accession: NP_001104754.1
IGF-1 isoform 1 preproprotein, 158 aa protein, Accession: NP_001104753.1
IGF-1 isoform 4 preproprotein, 153 aa protein, Accession: NP_000609.1

IL-4 (human)

### Interleukin-4, UniProtKB/Swiss-Prot: P05112.1, 153 aa

Interleukin-4 isoform 3 precursor, NP_001341919.1, 136 aa
Interleukin-4 isoform 2 precursor, NP_758858.1, 137 aa
Interleukin-4 isoform 1 precursor, NP_000580.1, 153 aa

### Interleukin-4, GenBank: AAH70123.1, 153 aa

### Interleukin-4, GenBank: AAH67514.1, 153 aa

Insulin (Human)

Insulin, 110 aa protein, Accession: AAA59172.1
Insulin, partial, 94 aa protein, Accession: AEG19452.1
Insulin, 110 aa protein, Accession: AAN39451.1
Insulin, 107 aa protein, Accession: AAA59179.1
Insulin, partial, 59 aa protein, Accession: CAA08766.1
Insulin, 98 aa protein, Accession: ABI63346.1

IGF-II (IGF-2) (Human)

Insulin-like growth factor II, GenBank: AAB34155.1, 180 aa
Insulin-like growth factor II isoform 1 preproprotein, NCBI Reference Sequence: NP_000603.1, 180 aa
Insulin-like growth factor II isoform 2, 236 aa protein, Accession: NP_001121070.1

Results

To address potential cross-regulation among phagocytes, we initially tested a panel of eleven soluble mediators that have been linked to tissue repair, inflammation dampening, and tissue morphogenesis, for their ability to modulate engulfment by LR73 fibroblasts, a non-professional phagocytic cell line. Of the eleven factors tested, only insulin-like growth factor I (IGF-I) significantly dampened apoptotic cell uptake, at concentrations reported in mouse and human serum (100-600 ng/mL) (FIG. 1a, 1b). Other factors such as EGF, FGF2, VEGF, PDGF-AA and PDGF-BB did not alter engulfment even over a range of concentrations (FIG. 5a), although they all elicited early downstream signaling events (FIG. 5b-e). The engulfment dampening effect of IGF-1 was also seen with airway epithelial cell lines BEAS-2B and 16HBE14o-, and the endothelial cell line SVEC-40 (FIG. 1c, Fig. f, g).

The IGF-1 effect was not due to masking phosphatidylserine (PtdSer) on the apoptotic cells (FIG. 1d). LR73 cells express the IGF-1 receptor (IGF-1R) and IGF-1 treatment elicited phosphorylation of Akt, a signaling molecule downstream of IGF-1R (FIG. 1b). In blood, IGF-1 is bound to IGF binding proteins (IGFBPs) that stabilize and sequester IGF-1. Addition of IGFBP3 with IGF-1 restored the phagocytic capability of LR73 cells (FIG. 1e), suggesting that binding of active IGF-1 to IGF-1R was necessary. A neutralizing antibody against the human IGF-1R reversed the engulfment-dampening effect of IGF-1 on BEAS-2B cells (FIG. 1f). Further, OSI-906 (a small molecule kinase inhibitor of IGF-1R currently tested in clinical trials), and another inhibitor, NVP-AEW541, rescued the engulfment capacity of LR73 cells treated with IGF-1, with concomitant decrease in phosphorylation of IGF-1R and Erk (FIG. 1g and FIG. 6a). IGF-II and insulin, which share structural similarity with IGF-1 but have lower affinities for IGF-1R, could also reduce apoptotic cell uptake (FIG. 6b, c), albeit at higher concentrations. Thus, productive signaling through the IGF-1R is necessary for IGF-1 to dampen apoptotic cell uptake.

In contrast to the inhibitory effect on apoptotic cell uptake, IGF-1 enhanced the uptake of PtdSer containing liposomes of 150-200 nm in size (FIG. 1h). This was not seen with EGF or VEGF. IGF-1 enhancement required IGF-1R signaling, as the inhibitor OSI-906 reduced the increased liposome uptake (FIG. 1i). Thus, IGF-1 can redirect phagocytosis by non-professional phagocytes, suppressing uptake of larger apoptotic cells while enhancing internalization of smaller particles. The IGF-1 effect was reversible, as washing phagocytes pre-treated with IGF-1 resulted in near complete restoration of engulfment (FIG. 1j). The effect of IGF-1 on phagocytes was also rapid, as adding IGF-1 simultaneously with apoptotic cells inhibited engulfment similar to pre-treated cells (data not shown), suggesting interference at early step(s) during phagocytosis. Blocking some early signaling pathways downstream of IGF-1R, such as Akt, mTOR, Erk or PI3-kinase, did not rescue the IGF-1-mediated decrease in corpse uptake (FIG. 7a-d). Although IGF-1 can activate RhoA, which is known to decrease apoptotic cell engulfment, inhibiting RhoA-mediated signaling did not reverse IGF-1-mediated engulfment suppression (FIG. 7e,f). Overexpressing activated Racl (which promotes apoptotic cell engulfment) bypassed attenuation of apoptotic cell uptake (FIG. 1k), suggesting that IGF-1R acts at or before a step regulated by active Racl. Since apoptotic cell uptake requires both Racl-dependent actin polymerization and de-polymerization, we explored actin regulation via IGF-1. Cytochalasin D (cytoD), which promotes actin depolymerization, potently inhibited phagocytosis of apoptotic cells (data not shown). While cytoD did not affect the basal uptake of liposomes by LR73 cells, it blocked the IGF-1 induced increase in liposome uptake (FIG. 1b. Of note, cytoD treated LR73 cells appeared morphologically normal for the duration of the assay. Latrunculin A, which promotes actin depolymerization via a different mechanism, also reversed IGF-1 mediated enhancement of liposome uptake, without affecting the basal uptake of liposomes (FIG. 1m). Additionally, Arp2/3 complex can regulate phagocytosis through the formation of branched actin networks; however, CK-666, a small molecule inhibitor of Arp2/3, had minimal effect on the IGF-1 mediated increase of liposome uptake (FIG. 7g). Collectively, IGF-1 mediated modulation of phagocytosis involves rapid and reversible modification of F-actin/G-actin dynamics, but likely not Arp2/3 mediated functions.

We next tested the effect of IGF-1 on professional phagocytes. Adding IGF-1 to the macrophage cell lines J774 or IC-21, or primary macrophages (bone marrow-derived or resident peritoneal macrophages), did not affect corpse uptake, even with supra-physiological concentrations of IGF-1, at every time point analyzed (FIG. 1n p, and FIG. 8a, d). Peritoneal macrophages exposed to IGF-1 also had comparable liposome uptake as control treated cells (FIG. 1q). Importantly, IGF-1R expression was confirmed in all macrophage cell types analyzed (FIG. 8b) and IGF-1 initiated early signaling in macrophages, as assessed by IGF-1R and Akt phosphorylation (FIG. 1n, FIG. 8a). These data suggest that IGF-1 mediated modulation of phagocytosis does not extend to macrophages.

High levels of serum IGF-1 is linked to IGF-1 production in the liver, but macrophages can also produce IGF-1 in response to the cytokine IL-4, raising the possibility that within a tissue, IGF-1 release from macrophages could regulate nearby non-professional phagocytes. We first confirmed IGF-1 secretion by peritoneal macrophages treated with IL-4 (FIG. 2a). Furthermore, resident peritoneal macrophages exposed to apoptotic Jurkat cells (but not live cells) produced IGF-1 (FIG. 2a). IGF-1 protein induction appears to be from newly transcribed Igf1 message (FIG. 9a). Macrophage produced IGF-1 could also suppress apoptotic cell uptake by LR73 cells, reversed by IGFBP3 addition (FIG. 2b).

To test the communication between phagocytes in vivo, we chose the lung, as alveolar macrophages and airway epithelial cells reside in close proximity, and both engulf apoptotic cells³. Alveolar macrophages, similar to resident peritoneal macrophages, are derived from fetal monocytes, and are readily isolated via CD11c⁺ Siglec F⁺ expression, while the airway epithelial cells can be isolated and tracked with available genetic tools. IGF-1R expression was prominent in the airway epithelial cells (FIG. 9b, c) but was also detectable at lower levels in alveolar macrophages. To test the effect of IGF-1 in vivo, we used mice where airway epithelial cells are specifically marked by YFP. Unexpectedly, when apoptotic cells or liposomes were administered intranasally with IGF-1 (FIG. 2c), YFP airway epithelial cells had decreased apoptotic cell engulfment, but enhanced lipo some uptake (FIG. 2d, e). Importantly, lung macrophages from the same IGF-1 treated mice were unaffected in their ability to engulf these targets (FIG. 2d, e). Thus, IGF-1 can differentially affect engulfment by professional and non-professional phagocytes closely residing in the same tissue.

We next examined IGF-1 release by alveolar macrophages in vivo. Intranasal administration of IL-4 or apoptotic Jurkat cells resulted in an increased level of IGF-1 in the bronchoalveolar lavage (BAL) fluid (FIG. 2f). This indicated that IGF-1 could be used as an agent for regulating inflammation in the lung. When we tested IL-13 or IL-5 (cytokines linked to lung homeostasis and inflammation), IL-13 (whose receptor shares a common subunit with the IL-4R), but not IL-5, induced IGF-1 production (FIG. 2f). Since the source of IGF-1 is difficult to distinguish in these experiments, we generated LysM-Cre/Igf1^{fl/fl} mice, targeting IGF-1 deletion in the myeloid lineage. LysM-Cre/Igf1^{fl/fl} mice showed loss of Igf1 mRNA in alveolar macrophages (FIG. 9d) and IGF-1 induction after IL-4, IL-13 or apoptotic cell treatment (FIG. 2g). This suggested the macrophage/myeloid population as the predominant source of inducible IGF-1 in the lung.

To investigate IGF-I/IGF-IR signaling in inflammation, we used a model of airway inflammation induced by the allergen house dust mite (HDM), where apoptotic cell recognition by airway epithelial cells influences inflammation³. We administered HDM intranasally in the allergen sensitization and challenge stage, modeling the natural route of allergen encounter (FIG. 3a). When we first tested LysM-Cre/Igf1^{fl/fl} mice, these mice proved unsuitable, as we continued to detect variable levels of IGF-I in the BAL fluid after HDM administration (likely due to leakage of serum IGF-I during inflammation). Therefore, we targeted instead the IGF-IR on the epithelial cells. We crossed Igf1r^{fl/fl} mice with CCSP-_{ntTA/tet( )}-Cre transgenic mice, the latter driving Cre under the Club cell secretory protein (CCSP) promoter in the epithelial cells of the trachea, bronchi and bronchioles. This CCSP-rtTA/tetO-Cre strain allows for inducible Cre expression through doxycycline administration via drinking water, thereby allowing normal development and gene deletion just prior to allergen exposure. We observed near complete loss of IGF-IR on epithelial cells of CCSP-Cre/Igf1r^{fl/fl} mice after doxycycline treatment (FIG. 3b). After sensitization and challenge with low-endotoxin HDM, the CCSP-Cre/Igf1r^{fl/fl} mice had greater airway inflammation based on several parameters. First, there was marked increase in eosinophils and CD4⁺ T-cells in the BAL fluid (FIG. 3c) and trending increase in inflammatory cells in the lungs (FIG. 10a). Second, lung-draining lymph nodes were larger with more CD4⁺ T-cells (FIG. 3d). Third, lung sections showed increased peribronchial and perivascular cellular infiltration (FIG. 3e, f) and greater mucus accumulation after HDM treatment (FIG. 3g, h). Fourth, HDM-treated CCSP-Cre/Igf1r^{fl/fl} mice displayed increased airway reactivity after methacholine challenge, a measure of the bronchial hyper-responsiveness (FIG. 10b). There were more apoptotic cells in lung sections (cleaved caspase 3 staining) in HDM-treated CCSP-Cre/Igf1r^{fl/fl} mice, likely due to greater inflammation (FIG. 10c). These data suggested a requirement for IGF-1R in the airway epithelial cells in minimizing inflammation in this model.

These observations were initially surprising, as we were expecting the loss of IGF-IR on airway epithelial cells to improve apoptotic cell clearance and thereby attenuate, rather than worsen, inflammation. This prompted us to examine the temporal requirement of IGF-I/IGF-IR signaling in epithelial cells during allergen exposure. To distinguish the requirement for IGF-I/IGF-IR signaling at the sensitization versus challenge phase, we administered doxycycline at different times: deleting IGF-IR expression before allergen sensitization (FIG. 4a), or after the initial allergen sensitization but before the challenge phase (FIG. 11a). Mice with IGF-1R deletion prior to allergen sensitization showed a significant increase in inflammatory parameters (FIG. 4b), while deleting IGF-1R after the sensitization phase had minimal effect on disease severity (FIG. 11b, c).

Airway epithelial cells encountering allergen can produce cytokines, such as TSLP, CSF-2/GM-CSF (affecting dendritic cell maturation), as well as IL-33 and IL-25 that drive type 2 innate lymphoid cells (ILC2s) to proliferate and produce IL-13. Bronchial epithelial cells from human asthmatics produce elevated levels of IL-6, IL-8, and CSF-2, and TSLP released from airway epithelial cells (mice and human) can exacerbate airway inflammation. When we assessed the cytokine profile in the BAL fluid of CCSP-Cre/Igf1r^{fl/fl} mice sensitized with HDM, levels of TSLP and IL-6 were greater (FIG. 4d, e). We did not find increased levels of IL-33, as seen with Racl deletion in airway epithelial cells³, but this may be due to differences in inflammatory pathways regulated by the loss of IGF-1R versus Racl. These results suggest that IGF-1R in airway epithelial cells limits inflammatory cytokines during the initial antigen exposure/sensitization phase.

As for trigger(s) for IGF-1 release from the alveolar macrophages in the sensitization phase, while IL-4 is often considered a classic Th2 cytokine from the adaptive immune response, both IL-4 and IL-13 can be produced during the allergen sensitization phase by resident mast cells, basophils, and/or ILC2s. In fact, IL-4 was enhanced (along with IL-5 and eotaxin-1) in the BAL fluid of CCSP-Cre/Igf1r^{fl/fl} mice sensitized with HDM (FIG. 4c). Thus, IL-4 and/or apoptotic cells present during the initial allergen exposure could elicit IGF-1 production from alveolar macrophages.

Alveolar macrophages are reported to release microvesicles containing anti-inflammatory mediators during smoking-induced lung injury. Since IGF-1 enhances liposome uptake by airway epithelial cells, we tested whether microvesicles from alveolar macrophages may impact airway epithelial cell response to HDM. Using differential centrifugation and filtration, we isolated and characterized microvesicles from alveolar macrophages (FIG. 4f). First, negative stain and cryo-electron microscopy revealed membrane bound spherical structures >100 nm in diameter (FIG. 4g, h). Second, via ImagestreamX™ analysis, particles from an alveolar macrophage cell line or primary mouse alveolar macrophages carry markers of lung macrophage origin, CD11c or Siglec F, with variable Annexin V staining (FIG. 4i). Third, tunable resistive pulse sensing analysis of microvesicles revealed a mean particle size of 357±148.5 nm (FIG. 4j), within the reported 100-1000 nm range for microvesicles. Further, IL-4 treatment of macrophages increased microvesicles secretion (FIG. 12a). Also, uptake of these microvesicles by BEAS-2B airway epithelial cells was enhanced by IGF-1 (FIG. 4k). Importantly, adding alveolar macrophage derived microvesicles to HDM-treated BEAS-2B cells resulted in significantly lower TSLP, CSF2, IL6, and IL8 induction (FIG. 4l). To further explore this, we performed RNA-seq of BEAS-2B cells treated with HDM±microvesicles. While HDM treatment increased several known genes associated with asthma in humans, such as FGF2, KLF4, Interferon-Induced Protein with Tetratricopeptide Repeats 2 (IFIT2), and Pentraxin 3 (PTX3), adding microvesicles from alveolar macrophages suppressed transcription of these genes in the epithelial cells (FIG. 4m, FIG. 1N)), Thus, microvesicles released from alveolar macrophages acting on airway epithelial cells exposed to allergen could dampen airway inflammation.

The data presented here provide several new insights on phagocytosis and tissue inflammation. First, it has long been known that professional and non-professional phagocytes reside in proximity and can engulf dying cells and debris, yet communication between these phagocytes was not understood. The data presented here identifies a rapid, transient, and reversible regulation, wherein soluble IGF-1 from macrophages influences the type of particle uptake by epithelial cells. This transient effect might allow the macrophages to temporarily ‘redirect’ the non-professional phagocytes towards other function(s). Such a possible hierarchy in cell clearance could provide temporal and spatial cross-communication within a given tissue. Second, our data identifies a two-part regulation of epithelial cells by macrophages which impacts airway inflammation, i.e., the secretion of IGF-1 that redirects particle uptake, and the release of microvesicles that dampen inflammatory cytokine production by epithelial cells (FIGS. 13a and 13b). Third, IGF-1 is a growth factor widely linked to growth, cellular proliferation, and aging. Global IGF-1 deletion in mice results in dwarfism and perinatal lethality, and IGF-1 mutations are linked to human diseases. This work identifies a previously unappreciated biological function for the IGF-1/IGF-1R axis as a modulator of airway hyper-responsiveness to allergens with potential therapeutic relevance.

The disclosures of each and every patent, patent application, and publication cited herein are hereby incorporated by reference herein in their entirety. The provisional patent application, from which this application claims priority, was based on a draft manuscript now published as Han et al., Nature, 2016, 539:570-74 entitled “Macrophages redirect phagocytosis by nonprofessional phagocytes and influence inflammation”.

Headings are included herein for reference and to aid in locating certain sections. These headings are not intended to limit the scope of the concepts described therein under, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

### BIBLIOGRAPHY

1. Henson, P. M. & Hume, D. A. Apoptotic cell removal in development and tissue homeostasis. Trends Immunol 27, 244-250, doi:10.1016/j.it.2006.03.005 (2006).
2. Burstyn-Cohen, T. et al. Genetic dissection of TAM receptor-ligand interaction in retinal pigment epithelial cell phagocytosis. Neuron 76, 1123-1132, doi:10.1016/j.neuron.2012.10.015 (2012).
3. Juncadella, I. J. et al. Apoptotic cell clearance by bronchial epithelial cells critically influences airway inflammation. Nature 493, 547-551, doi:10.1038/nature11714 (2013).
4. Wood, W. et al. Mesenchymal cells engulf and clear apoptotic footplate cells in macrophageless PU.1 null mouse embryos. Development 127, 5245-5252 (2000).
5. Lu, Z. et al. Phagocytic activity of neuronal progenitors regulates adult neurogenesis. Nat Cell Biol 13, 1076-1083, doi:10.1038/ncb2299 (2011).
6. Ichimura, T. et al. Kidney injury molecule-1 is a phosphatidylserine receptor that confers a phagocytic phenotype on epithelial cells. J Clin Invest 118, 1657-1668, doi:10.1172/JCI34487 (2008).
7. Mesa, K. R. et al. Niche-induced cell death and epithelial phagocytosis regulate hair follicle stem cell pool. Nature 522, 94-97, doi:10.1038/nature14306 (2015).
8. Elliott, M. R. et al. Unexpected requirement for ELMO1 in clearance of apoptotic germ cells in vivo. Nature 467, 333-337, doi:10.1038/nature09356 (2010).
9. Gregory, C. D. & Pound, J. D. Microenvironmental influences of apoptosis in vivo and in vitro. Apoptosis 15, 1029-1049, doi:10.1007/s10495-010-0485-9 (2010).
10. Green, D. R. The end and after: how dying cells impact the living organism. Immunity 35, 441-444, doi:10.1016/j.immuni.2011.10.003 (2011).
11. Werner, S. & Grose, R. Regulation of wound healing by growth factors and cytokines. Physiol Rev 83, 835-870, doi:10.1152/physrev.00031.2002 (2003).
12. Yakar, S. et al. Circulating levels of IGF-1 directly regulate bone growth and density. J Clin Invest 110, 771-781, doi:10.1172/JCI15463 (2002).
13. Smith, T. J. Insulin-like growth factor-I regulation of immune function:

a potential therapeutic target in autoimmune diseases? Pharmacol Rev 62, 199-236, doi:10.1124/pr.109.002469 (2010).
14. LeRoith, D. Insulin-like growth factor I receptor signaling—overlapping or redundant pathways? Endocrinology 141, 1287-1288, doi:10.1210/endo.141.4.7475 (2000).
15. Tosello-Trampont, A. C., Nakada-Tsukui, K. & Ravichandran, K. S.

Engulfment of apoptotic cells is negatively regulated by Rho-mediated signaling. J Biol Chem 278, 49911-49919, doi:10.1074/jbc.M306079200 (2003).
16. Leverrier, Y. & Ridley, A. J. Requirement for Rho GTPases and PI 3-kinases during apoptotic cell phagocytosis by macrophages. Curr Biol 11, 195-199 (2001).
17. Tosello-Trampont, A. C., Brugnera, E. & Ravichandran, K. S. Evidence for a conserved role for CRKII and Rac in engulfment of apoptotic cells. J Biol Chem 276, 13797-13802, doi:10.1074/jbc.M011238200 (2001).
18. Schlam, D. et al. Phosphoinositide 3-kinase enables phagocytosis of large particles by terminating actin assembly through Rac/Cdc42 GTPase-activating proteins. Nat Commun 6, 8623, doi:10.1038/ncomms9623 (2015).
19. Chen, F. et al. An essential role for TH2-type responses in limiting acute tissue damage during experimental helminth infection. Nat Med 18, 260-266, doi:10.1038/nm.2628 (2012).
20. Ginhoux, F. & Guilliams, M. Tissue-Resident Macrophage Ontogeny and Homeostasis. Immunity 44, 439-449, doi:10.1016/j.immuni.2016.02.024 (2016).
21. Lambrecht, B. N. & Hammad, H. The immunology of asthma. Nat Immunol 16, 45-56, doi:10.1038/ni.3049 (2015).
22. Marini, M., Vittori, E., Hollemborg, J. & Mattoli, S. Expression of the potent inflammatory cytokines, granulocyte-macrophage-colony-stimulating factor and interleukin-6 and interleukin-8, in bronchial epithelial cells of patients with asthma. J Allergy Clin Immunol 89, 1001-1009 (1992).
23. Zhou, B. et al. Thymic stromal lymphopoietin as a key initiator of allergic airway inflammation in mice. Nat Immunol 6, 1047-1053, doi:10.1038/ni1247 (2005).
24. Gessner, A., Mohrs, K. & Mohrs, M. Mast cells, basophils, and eosinophils acquire constitutive IL-4 and IL-13 transcripts during lineage differentiation that are sufficient for rapid cytokine production. J Immunol 174, 1063-1072 (2005).
25. Sokol, C. L., Barton, G. M., Fan, A. G. & Medzhitov, R. A mechanism for the initiation of allergen-induced T helper type 2 responses. Nat Immunol 9, 310-318, doi:10.1038/ni1558 (2008).
26. Bourdonnay, E. et al. Transcellular delivery of vesicular SOCS proteins from macrophages to epithelial cells blunts inflammatory signaling. J Exp Med 212, 729-742, doi:10.1084/jem.20141675 (2015).
27. Burger, D. et al. Microparticles: biomarkers and beyond. Clin Sci (Lond) 124, 423-441, doi:10.1042/CS20120309 (2013).
28. Bissonnette, E. Y. et al. Fibroblast growth factor-2 is a sputum remodeling biomarker of severe asthma. J Asthma 51, 119-126, doi:10.3109/02770903.2013.860164 (2014).
29. Zhang, J. et al. Pentraxin 3 (PTX3) expression in allergic asthmatic airways: role in airway smooth muscle migration and chemokine production. PLoS One 7, e34965, doi:10.1371/journal.pone.0034965 (2012).
30. Chamberland, A., Madore, A. M., Tremblay, K., Laviolette, M. & Laprise, C. A comparison of two sets of microarray experiments to define allergic asthma expression pattern. Exp Lung Res 35, 399-410 (2009).
31. Perl, A. K., Zhang, L. & Whitsett, J. A. Conditional expression of genes in the respiratory epithelium in transgenic mice: cautionary notes and toward building a better mouse trap. Am J Respir Cell Mol Biol 40, 1-3, doi:10.1165/rcmb.2008-0011ED (2009).
32. Dietrich, P., Dragatsis, I., Xuan, S., Zeitlin, S. & Efstratiadis, A. Conditional mutagenesis in mice with heat shock promoter-driven cre transgenes. Mamm Genome 11, 196-205 (2000).
33. Liu, J. L., Yakar, S. & LeRoith, D. Conditional knockout of mouse insulin-like growth factor-1 gene using the Cre/loxP system. Proc Soc Exp Biol Med 223, 344-351 (2000).
34. Liu, J. L. et al. Insulin-like growth factor-I affects perinatal lethality and postnatal development in a gene dosage-dependent manner: manipulation using the Cre/loxP system in transgenic mice. Mol Endocrinol 12, 1452-1462, doi:10.1210/mend.12.9.0162 (1998).
35. Hahn, Y. S. et al. V gamma 4+ gamma delta T cells regulate airway hyperreactivity to methacholine in ovalbumin-sensitized and challenged mice. J Immunol 171, 3170-3178 (2003).
36. Yeager, M., Berriman, J. A., Baker, T. S. & Bellamy, A. R. Three-dimensional structure of the rotavirus haemagglutinin VP4 by cryo-electron microscopy and difference map analysis. EMBO J 13, 1011-1018 (1994).
37. Casella, J. F., Flanagan, M. D. & Lin, S. Cytochalasin D inhibits actin polymerization and induces depolymerization of actin filaments formed during platelet shape change. Nature 293, 302-305 (1981).
38. Yarmola, E. G., Somasundaram, T., Boring, T. A., Spector, I. & Bubb, M. R. Actin-latrunculin A structure and function. Differential modulation of actin-binding protein function by latrunculin A. J Biol Chem 275, 28120-28127, doi:10.1074/jbc.M004253200 (2000).
39. Hetrick, B., Han, M. S., Helgeson, L. A. & Nolen, B. J. Small molecules CK-666 and CK-869 inhibit actin-related protein 2/3 complex by blocking an activating conformational change. Chem Biol 20, 701-712, doi:10.1016/j.chembio1.2013.03.019 (2013).
40. Mulvihill, M. J. et al. Discovery of OSI-906: a selective and orally efficacious dual inhibitor of the IGF-1 receptor and insulin receptor. Future Med Chem 1, 1153-1171, doi:10.4155/fmc.09.89 (2009).
41. Garcia-Echeverria, C. et al. In vivo antitumor activity of NVP-AEW541-A novel, potent, and selective inhibitor of the IGF-IR kinase. Cancer Cell 5, 231-239 (2004).

What is claimed is:

1. A method for decreasing an inflammatory response in non-professional phagocytes in a subject in need thereof, the method comprising administering to the subject a pharmaceutical composition comprising an effective amount of macrophage-derived microvesicles or stimulating macrophages in the subject to secrete Insulin-like Growth Factor-I (IGF-I) and to release microvesicles, wherein uptake of the microvesicles by the non-professional phagocytes decreases production and release of one or more inflammatory response associated proteins by the non-professional phagocytes, and the secreted IGF-I enhances uptake of the microvesicles into the non-professional phagocytes, thereby decreasing the inflammatory response in non-professional phagocytes.

2. The method of claim 1, wherein the non-professional phagocytes are epithelial cells.

3. The method of claim 2, wherein the epithelial cells are airway epithelial cells.

4. The method of claim 3, wherein the inflammatory response of the airway epithelial cells is initiated by an allergen contacting the airway epithelial cells.

5. The method of claim 1, wherein the inflammatory response associated proteins are selected from the group consisting of thymic stromal lymphopoietin (TSLP), colony stimulating factor 2/granulocyte-macrophage colony stimulating factor (CSF-2/GM-CSF), interleukin 6 (IL-6), interleukin 8 (IL-8), interleukin 25 (IL-25), interleukin 33 (IL-33), fibroblast growth factor 2 (FGF2), Kruppel-like factor 4 (KLF4), Interferon-Induced Protein with Tetratricopeptide Repeats 2 (IFIT2), and Pentraxin 3 (PTX3).

6. The method of claim 1, wherein an effective amount of Interleukin-4 (IL-4) or Interleukin-13 (IL-13) is administered to the subject to stimulate the macrophages to release macrophage vesicles and IGF-I.

7. The method of claim 1, wherein the method stimulates an increase in macrophage microvesicle uptake by the non-professional phagocytes.

8. The method of claim 1, wherein the methods inhibits uptake of apoptotic cells by the non-professional phagocytes.

9. The method of claim 1, wherein the macrophage-derived microvesicles administered to the subject are purified macrophage-derived microvesicles.

10. The method of claim 1, wherein an effective amount of IGF-I is administered to the subject to increase macrophage microvesicle uptake by the non-professional phagocytes.

11. The method of claim 1, wherein the inflammatory response is inflammatory cytokine production.

12. A method for decreasing an inflammatory response in non-professional phagocytes in a subject in need thereof, the method comprising administering to the subject a pharmaceutical composition comprising an effective amount of Insulin-like Growth Factor I (IGF-I), macrophage-derived microvesicles, insulin, or Insulin-like Growth Factor-II, wherein the method decreases uptake of apoptotic cells by the non-professional phagocytes, thereby decreasing an inflammatory response in non-professional phagocytes.

13. The method of claim 12, wherein the decrease in the inflammatory response in the non-professional phagocytes is a decrease in one or more inflammatory response associated proteins in the non-professional phagocytes.

14. The method of claim 13, where the inflammatory response associated proteins are selected from the group consisting of thymic stromal lymphopoietin (TSLP), colony stimulating factor 2/granulocyte-macrophage colony stimulating factor (CSF-2/GM-CSF), interleukin 6 (IL-6), interleukin 8 (IL-8), interleukin 25 (IL-25), interleukin 33 (IL-33), fibroblast growth factor 2 (FGF2), Kruppel-like factor 4 (KLF4), Interferon-Induced Protein with Tetratricopeptide Repeats 2 T′2), and Pentraxin 3 (PTX3).

15. The method of claim 12, wherein an effective amount of IL-4 is administered to the subject to stimulate macrophages in the subject to release macrophage vesicles and IGF-I.

16. The method of claim 12, wherein the method stimulates an increase in macrophage microvesicle uptake by the non-professional phagocytes.

17. The method of claim 12, wherein macrophage-derived microvesicles that are administered to the subject are purified macrophage-derived microvesicles.

18. The method of claim 12, wherein an effective amount of IGF-I is administered to the subject to increase macrophage microvesicle uptake by the non-professional phagocytes.

19. The method of claim 12, wherein uptake of the microvesicles by the non-professional phagocytes decreases production and release of one or more inflammatory response associated proteins by the non-professional phagocytes.

20. The method of claim 12, wherein the non-professional phagocytes are epithelial cells.

21. The method of claim 20, wherein the epithelial cells are airway epithelial cells.

22. The method of claim 21, wherein the inflammatory response is initiated by an allergen contacting the airway epithelial cells.

23. The method of claim 12, wherein the inflammatory response is inflammatory cytokine production.

24. A method to decrease an inflammatory response in an epithelial cell, the method comprising contacting the epithelial cell with an effective amount of Insulin-like Growth Factor I (IGF-I), macrophage-derived microvesicles, insulin, or Insulin-like Growth Factor-II, wherein the method decreases uptake of apoptotic cells.

25. The method of claim 24, wherein uptake of macrophage-derived microvesicles by the epithelial cell is increased by contacting the epithelial cell with IGF-I.

26. The method of claim 24, wherein the epithelial cell is an airway epithelial cell.

27. The method of claim 26, wherein the inflammatory response is initiated by exposure of the epithelial cell to an allergen.

28. The method of claim 24, wherein the macrophage-derived microvesicles are purified, and then the epithelial cell is contacted with the purified macrophage-derived microvesicles.

29. The method of claim 24, wherein the macrophage-derived microvesicles have been released by macrophages stimulated to release the microvesicles.

30. The method of claim 29, wherein the macrophages are stimulated to release the microvesicles by contact with Interleukin-4.

31. The method of claim 29, wherein the macrophages are in close proximity to the epithelial cell.
